SEQUENCE LISTING (36 sequences)

SEQ ID NO: 1 - DNA, 3726 bp, Homo sapiens
  CDS (1)..(2484): human XT-I     (not completely)
actcaggatggctacttttctcatcggccgaaagagaaagtgcgaacagacagcaacaacgagaactctgtccccaaagactttgagaatgtggacaacagcaacttcgcacccaggactcaaaagcagaagcaccagcctgagttggcgaagaagccaccgagtagacagaaggagcttttgaaaaggaagctggaacagcaggagaaaggaaaaggacatacattccctgggaaaggccccggtgaggtgctgcctcccggggacagagccgcagccaacagcagccacgggaaggatgtgtccagaccgcctcatgccaggaaaactgggggcagctcccccgagaccaagtatgaccagccccctaagtgtgacatctcaggcaaggaggccatctctgccctgtcccgtgctaagtccaagcactgccgccaggagattggggagacttactgccgccacaagttagggctgctgatgcctgagaaggtgactcggttctgccccctcgagggtaaagccaacaagaacgtgcagtgggacgaggactccgtggagtacatgccagccaacccggtcagaatcgcctttgtcctggtggtccacggccgtgcctctcggcagttgcagcgcatgttcaaggccatctaccacaaagaccacttctactacatccacgtggacaagcgctctaattacctgcatcggcaagtgctccaggtctccaggcagtacagcaatgtccgcgtcaccccctggagaatggccaccatctggggaggagccagcctcctgtccacctacctgcagagcatgcgggacctcctggagatgaccgactggccctgggacttcttcatcaacctgagtgcggccgactaccccatcaggacaaatgaccagttggtggcgtttctctcccgataccgagatatgaatttcttgaagtcacacggccgggacaatgcaaggttcattcggaagcagggcctggatcggctcttcctggagtgcgacgctcacatgtggcgcctgggagatcggcggatcccagagggcattgccgtggatggcggttcggactggttcctgctgaaccggaggtttgtagaatatgtgaccttctccacagacgatctggtgaccaagatgaaacagttctactcctacaccctgcttcctgctgagtccttcttccatacggtcctggagaacagcccccactgcgacaccatggtggacaacaacctgcgcatcaccaactggaatcgcaagctgggctgcaagtgccagtacaagcacatcgtggactggtgcggctgctcccccaatgacttcaagccgcaggacttccaccgcttccagcagacagcccggcctaccttctttgcccgcaagtttgaagccgtggtgaatcaggaaatcattgggcagctggactattacctgtacgggaactaccctgcaggtaccccgggcctgcgctcctactgggagaatgtctacgatgagcctgacggcatccacagcctgagcgacgtgacactcaccttgtaccactcctttgcccgcctgggtcttcgacgggctgagacgtccctgcacacggatggggagaacagctgccgatactacccaatgggccacccagcatctgtgcacctctacttccttgctgaccgcttccagggctttctgatcaagcatcatgctaccaatctggctgtgagcaaactagagactctggagacctgggtgatgccgaaaaaagtcttcaagatcgcaagcccacccagtgactttgggaggcttcagttttccgaggtcggcactgactgggatgccaaggagaggctattccgcaactttgggggtcttctggggcccatggatgagccggtgggtatgcagaagtgggggaagggacctaatgtgaccgtgaccgtcatttgggtggatcccgtcaatgtcatcgcagccacctacgacatcctcattgagtccactgccgaattcacacactacaagccccctttgaacttgcccctgaggcctggggtctggacagtgaaaattctccaccactgggtgccagttgcagagaccaaattcctcgttgcgcctctgaccttctcgaacaggcagcccatcaaacctgaggaggcactgaagctgcacaatgggcccctccgcaatgcctacatggagcagagcttccagagcctaaaccccgtcctcagcctgcccatcaaccccgcccaggtggaacaggcacggaggaacgcagcctccacgggcacagcgctggagggatggctggactcgttggtgggcgggatgtggactgccatggacatctgtgccacgggccccacagcctgcccggtcatgcagacctgcagccagacggcctggagctccttcagccctgaccccaagtcggagctgggtgccgtcaaacctgatggccggctcaggtagcactgggcacgaggagtgggccacagcaggatctcaacgggaaagcagccagaggggttgtggggcctgaaccccggcctccaccctgggggaggccctctgtgaatgggtctctcctggccatagaatgatggaaagggaaggtcagcaggtcaaagcaggatcagccaacaacctgcctttggcaagctgcgggtgggatggctcagtccctgcactgtgactgtctcacctcttctgsttgatcctcaagtcctacaggttccttgtcttccccttccagtgacccacccctgaccccagacgtgtgattttcagacttttctttcgagcagcagaacttcgtttacggagcacagtcataagtgraggttcagggtgctgacgaaatccaagctgctctggttgaagctgacaagtgcgaggttccctcccaaagctcagccctctgggcggtccccttgcccagggtatctcctacggtacctcttcagaacccaagggctctgcaaatgccagtttgacaagcactgcccagaccaaccatgggttcaaactccagccctgccctttggttcatttttctgcttctcttggctgggggactctggtgccagccttgaaagtcatggtcgtgggccctttcccatggaggctgcagccttaggagagctctgagcctctcagcagccctccttgggttgaactattctccttagtaactaggtaagtgggaaagccttttgatgtggcatggccaaggtccagccacaagtgcaactgccacctgtccaggggtctgggcctccttccctcaaggctgccacacaaagtagcagaaataggatgatgtttgtgagcaccagactcaagaccatgaccttctttgatccttgaaaatgggaactttgacagccatgaccatgaaactcacaaggcaacgcggatgaaactcacaaagcaatgcttggagcaaaactcctgagctagacagcacagcagcacccatcccctgccagagcccttccgtttgaggtcagacacacaaaaccttcgtcaattgcacaccggtgctgttgggagtgaccaaaccacatgaaccagacttttcccgtccaggaaatagcatttcagatttggtttttaatttcatgcccttcggccacaggctcaacgggacatgcaacataaaaatgggaaggttattcaacccgcgggaaaaaaaaaaaaaaaaaaaaaaaa

SEQ ID NO: 2 - protein, 827 aa, Homo sapiens
TQDGYFSHRPKEKVRTDSNNENSVPKDFENVDNSNFAPRTQKQKHQPELAKKPPSRQKELLKRKLEQQEKGKGHTFPGKGPGEVLPPGDRAAANSSHGKDVSRPPHARKTGGSSPETKYDQPPKCDISGKEAISALSRAKSKHCRQEIGETYCRHKLGLLMPEKVTRFCPLEGKANKNVQWDEDSVEYMPANPVRIAFVLVVHGRASRQLQRMFKAIYHKDHFYYIHVDKRSNYLHRQVLQVSRQYSNVRVTPWRMATIWGGASLLSTYLQSMRDLLEMTDWPWDFFINLSAADYPIRTNDQLVAFLSRYRDMNFLKSHGRDNARFIRKQGLDRLFLECDAHMWRLGDRRIPEGIAVDGGSDWFLLNRRFVEYVTFSTDDLVTKMKQFYSYTLLPAESFFHTVLENSPHCDTMVDNNLRITNWNRKLGCKCQYKHIVDWCGCSPNDFKPQDFHRFQQTARPTFFARKFEAVVNQEIIGQLDYYLYGNYPAGTPGLRSYWENVYDEPDGIHSLSDVTLTLYHSFARLGLRRAETSLHTDGENSCRYYPMGHPASVHLYFLADRFQGFLIKHHATNLAVSKLETLETWVMPKKVFKIASPPSDFGRLQFSEVGTDWDAKERLFRNFGGLLGPMDEPVGMQKWGKGPNVTVTVIWVDPVNVIAATYDILIESTAEFTHYKPPLNLPLRPGVWTVKILHHWVPVAETKFLVAPLTFSNRQPIKPEEALKLHNGPLRNAYMEQSFQSLNPVLSLPINPAQVEQARRNAASTGTALEGWLDSLVGGMWTAMDICATGPTACPVMQTCSQTAWSSFSPDPKSELGAVKPDGRLR

SEQ ID NO: 3 - DNA, 3608 bp, Homo sapiens
  CDS (150)..(2744): human XT-II
cgcaggccccgcccccggccccgccccggcgcgcgcccggcccgcctccctaggcgtggaggagggggggcggctcagccccgcgcccgtgcgcgcgctcgcggccgggttgcagggctgggcgcgcgccccgcgtcccgggcaggaagatggtggcgagcgcgcgagtgcagaagctggtgcggcgctacaagctggcgattgccacggcgctggccatcctgctgctgcagggcctggtagtgtggagcttcagcggcctggaggaggacgaggcgggcgagaaaggaaggcagaggaagccacggccactggaccctggcgaaggttccaaggacacagacagttcagcagggcgacggggcagcacaggcagaaggcatgggcgctggcggggccgtgctgagagcccaggagtgcccgtggccaaggtggtacgggcagtaaccagccggcagagagccagccggcgggtcccacctgccccacctccggaagccccaggccgccagaacctgagtggggcagcagctggggaggcgctggtaggggcagctggcttcccaccacacggagatacagggagcgtggagggcgccccccagcccacggacaatggcttcacccccaagtgcgagatcgtgggcaaggacgcactgtctgcactggcccgggccagcaccaagcagtgccagcaggagatcgccaatgtggtgtgcctgcaccaggctgggagcctcatgcccaaggctgtgccccggcactgtcagctgactgggaagatgagccccggcatccagtgggatgagagccaagcccagcagcccatggatggccccccggtgcgaatcgcctacatgctggtggttcacggccgcgccatccgccagctgaagcgtctcctcaaggccgtttatcacgagcagcacttcttttacatccatgtggacaagcgttccgactacctgcaccgggaggtggtggagctggcccagggctatgataacgtgcgggtgacgccctggcgcatggttaccatctggggcggggccagcctcctgacgatgtacctgcggagcatgcgggacctgctagaggtgcctggctgggcctgggacttcttcatcaacctcagtgccactgactatccaaccaggaccaatgaggagctggtggcattcctatccaagaaccgggacaagaatttcctcaagtcacatggccgggacaactccaggttcatcaagaaacagggcctggaccggctcttccatgagtgcgactcacacatgtggcgcctgggcgagcggcagatcccagcaggcattgtggtggatggcggttctgactggttcgtgctgacacgcagctttgtggagtatgtggtgtacacagatgacccgcttgtggcccagctgcgccagttctacacatacacactgctcccagccgagtccttcttccacacggtgctggagaacagcctggcctgtgagaccctcgtggacaacaacctgcgggtcaccaactggaaccgcaagctgggctgcaagtgccagtacaagcacattgtggactggtgtggctgctcccccaacgacttcaagccacaggacttcctccggctgcagcaagtctccagacccaccttcttcgcccggaagttcgagtcgactgtgaaccaggaggtgctggaaatcctggacttccacctgtacggcagctacccccccggcacgccagccctcaaggcctactgggagaacacctacgacgcggctgatggccccagtgggctcagtgatgtcatgctcactgcttacacagccttcgcccgcctcagcctgcaccatgccgccactgctgcacccccaatgggcaccccactctgcaggtttgagcccaggggcttgccgtccagcgtgcacctgtatttctatgacgaccatttccagggctacctggtgacgcaggcggtgcagccctcagcccaggggccggcagagacgcttgagatgtggctgatgccccaagggtcgctgaagctgttggggcgcagtgaccaggccagccggctccagagtctggaggttggcactgattgggaccccaaagagcgtcttttccggaactttggggggttactggggccgctggacgagcctgtggccgtgcagcgctgggcccggggccccaacctcacagccacagtggtctggatcgacccaacctatgtggtggccacatcttatgacatcacagtagatacggagactgaggtcacgcaatacaagcccccactgagccggcccctgcggccagggccctggactgttcgactccttcagttctgggaaccgctgggtgagacccgcttccttgtgctgcccttgaccttcaaccgcaaactacctctcaggaaagatgatgccagctggctgcacgcagggccaccccacaacgagtacatggagcagagtttccagggcctgagtagcatcctgaacctgcctcagccggagctcgcggaggaggctgcccagcggcacacacagctcacaggccctgcgctcgaggcctggacagacagggaactgagcagcttctggtccgtggctggactgtgtgccataggcccctctccctgcccctccctggagccctgcagactgaccagctggagctctctgtcccccgaccccaaatcagagctggggcctgtcaaagcagacgggcgactcaggtagcagggccccagccagtacccgtggaggacccgggaaattgcaccttacagacagtggaggggtgtcccctcccacaggcaagaaccagaggcccaggctgcacacccatttcagccatcaagaacccacacagacggcagggaaggtggacacagtatgaactactgctgatgtctctgttggggatcagagggctggcgggaacgcgagaagggcaccagcagcattccacacccagctcttcctcaccttcctgtctagtttgaatttcttttttttctttcttttttttttttttttaatttaaaaaggaaaatgggtggttgggagagaaactagaacagaagatgtgcaatagggctcagagcagccccaggaagtgggccatgtctgtggggagcagtggcttgctgagcccacctgctattgttctgcacgaggctgggcctgcctcactctccagggcctcatgccccattctgggcctgtggtgctcgtggctgaggctccacagggctgcaagtgccctgccaggctctaaggcccgaagaacaggtgatatcgggggcgctgcagagctgggctctagcaggacagttcttttgtagccaggggaccctagaagtggggtggggcggctccagggctttccagcataccctgcccctggatgggaaaggcagggtcagggcccactgagacccatgcagagtttccaggtgtgcactggaagtgaggtcacatgagcagcgtgggaagaagactctgtcaagactctcagaagaacctggagtaattgtgcctgaagctcagcgtgaagtctgaaatatgcaacagaagaaatatatctctatctctcta

SEQ ID NO: 4 - protein, 865 aa, Homo sapiens
MVASARVQKLVRRYKLAIATALAILLLQGLVVWSFSGLEEDEAGEKGRQRKPRPLDPGEGSKDTDSSAGRRGSTGRRHGRWRGRAESPGVPVAKVVRAVTSRQRASRRVPPAPPPEAPGRQNLSGAAAGEALVGAAGFPPHGDTGSVEGAPQPTDNGFTPKCEIVGKDALSALARASTKQCQQEIANVVCLHQAGSLMPKAVPRHCQLTGKMSPGIQWDESQAQQPMDGPPVRIAYMLVVHGRAIRQLKRLLKAVYHEQHFFYIHVDKRSDYLHREVVELAQGYDNVRVTPWRMVTIWGGASLLTMYLRSMRDLLEVPGWAWDFFINLSATDYPTRTNEELVAFLSKNRDKNFLKSHGRDNSRFIKKQGLDRLFHECDSHMWRLGERQIPAGIVVDGGSDWFVLTRSFVEYVVYTDDPLVAQLRQFYTYTLLPAESFFHTVLENSLACETLVDNNLRVTNWNRKLGCKCQYKHIVDWCGCSPNDFKPQDFLRLQQVSRPTFFARKFESTVNQEVLEILDFHLYGSYPPGTPALKAYWENTYDAADGPSGLSDVMLTAYTAFARLSLHHAATAAPPMGTPLCRFEPRGLPSSVHLYFYDDHFQGYLVTQAVQPSAQGPAETLEMWLMPQGSLKLLGRSDQASRLQSLEVGTDWDPKERLFRNFGGLLGPLDEPVAVQRWARGPNLTATVVWIDPTYVVATSYDITVDTETEVTQYKPPLSRPLRPGPWTVRLLQFWEPLGETRFLVLPLTFNRKLPLRKDDASWLHAGPPHNEYMEQSFQGLSSILNLPQPELAEEAAQRHTQLTGPALEAWTDRELSSFWSVAGLCAIGPSPCPSLEPCRLTSWSSLSPDPKSELGPVKADGRLR

SEQ ID NO: 5 - DNA, 2463 bp, Rat coronavirus
  CDS (1)..(2463): rat XT-I
tacttctctcaccggcccaaagagaaagttcggaccgacagcaacaatgagaactcagtccccaaggactttgagaatgtggacaacagcaacttcgcacccaggactcaaaagcagaagcaccagccagagttggcaaagaagcccctcagcaggcagaaagagcgtttgcagagaaagctgggcgcccaggacaaaggacaggggcagtcagtcctaggaaaaggccccaaggaggtgctgcctcctcgggaaaaagccccaggcaacagtagccaagggaaggatctctcaagacacagccatagcaggaagagtggtggcggtgggtccccggaaactaagtctgaccaggtccccaagtgtgacatctctggcaaggaggccatctcagcgctgacccgcgctaagtccaagcactgtcgccaggagattgcagaaacctactgtcgccacaagctggggctgctgatgccagagaaggtggctcgattctgtcccctggaaggcaaagccaacaagaatgtccagtgggatgaggatgctgttgagtacatgccccccaacccagtcagaatcgcctttgtcctggtggtccatggccgtgcctctcgacagctgcagcgcatgtttaaggccatctaccacaaagaccatttctactatatccatgtggataagcgttccaattacctgcatcggcaagtgctccagttctccaggcagtacgacaacgtccgagtcacttcctggaggatggccaccatttggggcggagccagcctcctgtccacttacctgcagagcatgcgggatctactggagatgactgactggccctgggacttcttcatcaacctgagtgcggctgactaccccatcaggacaaatgaccagctcgtagcatttctttccagataccgagatatgaatttcctgaagtcacatggtcgggacaatgcaaggttcatccggaagcaggacctggaccgtctcttcctggagtgtgatacacacatgtggcgcctgggggaccggcgaatcccagagggcattgctgtagatggcggctctgattggttcctgctaaaccggaagtttgtagagtatgtggcattctccacagatgacctggtgaccaagatgaagcagttctactcttataccctcctccctgctgagtcctttttccacacggtcctggagaacagcccccactgtgacaccatggtggataacaacctgcgcatcaccaattggaaccgcaagctgggctgcaagtgccagtacaagcatattgtggactggtgtggctgctctcccaatgacttcaagcctcaggacttccatcgcttccagcagacggcccggcccaccttctttgcccgaaagttcgaagccatagtgaaccaggaaatcattgggcagttggactcttacctgtatggcaactatcctgcgggcaccccgggcctccgctcttactgggagaatgtctatgacgaaccagatggcatccagagcctcagcgatgtggcactcaccatgtatcattccttcatccgcctgggtcttcgaagggctgagtcatcgctacacacggatggggagaacagctgcaggtactatccaatgggccacccagcgtctgtccacctctacttcctggctgaccgattccagggctttctgatcaagcaccatgtgaccaaccttgctgtgagcaaactggagacactggagacatggatgatgccaaagaaagtcttcaaggtcgcaagtccccccagtgactttgggaggcttcagttttctgaggttggcactgactgggatgccaaggagagactattccggaactttggtggtcttttggggcccatggatgagccggtgggaatgcagaaatgggggaagggacccaatgtgaccgtgactgttatttgggtggatcctgtcaatgtcatcgcagccacctatgatatcctgatcgagtccactgcggaattcacacactacaaaccccctttgaatctgcctctgaggcctggtgtctggacagtgaagattctccatcactgggtaccagttgcagagaccaaattccttgtcgcacctctgaccttctccaacaagcagcccatcaaaccagaggaggctttgaagctgcacaatgggccacctcgcagtgcctacatggagcagagtttccagagcctaaacccagtcctcagcctgcacatcaatcctgcccaagtggagcaggcccggaagaacgcagccttcactgggacagcactagaagcctggctggtgggtgggacttggactgccatggacgtctgtgccacaggccccactgcctgcccagtcatgcaaacctgcagccaaacagcctggagttccttcagccctgatcccaagtcagagctgggtgcagtgaagcctgatggtcggctcagg

SEQ ID NO: 6 - protein, 821 aa, Rat coronavirus
YFSHRPKEKVRTDSNNENSVPKDFENVDNSNFAPRTQKQKHQPELAKKPLSRQKERLQRKLGAQDKGQGQSVLGKGPKEVLPPREKAPGNSSQGKDLSRHSHSRKSGGGGSPETKSDQVPKCDISGKEAISALTRAKSKHCRQEIAETYCRHKLGLLMPEKVARFCPLEGKANKNVQWDEDAVEYMPPNPVRIAFVLVVHGRASRQLQRMFKAIYHKDHFYYIHVDKRSNYLHRQVLQFSRQYDNVRVTSWRMATIWGGASLLSTYLQSMRDLLEMTDWPWDFFINLSAADYPIRTNDQLVAFLSRYRDMNFLKSHGRDNARFIRKQDLDRLFLECDTHMWRLGDRRIPEGIAVDGGSDWFLLNRKFVEYVAFSTDDLVTKMKQFYSYTLLPAESFFHTVLENSPHCDTMVDNNLRITNWNRKLGCKCQYKHIVDWCGCSPNDFKPQDFHRFQQTARPTFFARKFEAIVNQEIIGQLDSYLYGNYPAGTPGLRSYWENVYDEPDGIQSLSDVALTMYHSFIRLGLRRAESSLHTDGENSCRYYPMGHPASVHLYFLADRFQGFLIKHHVTNLAVSKLETLETWMMPKKVFKVASPPSDFGRLQFSEVGTDWDAKERLFRNFGGLLGPMDEPVGMQKWGKGPNVTVTVIWVDPVNVIAATYDILIESTAEFTHYKPPLNLPLRPGVWTVKILHHWVPVAETKFLVAPLTFSNKQPIKPEEALKLHNGPPRSAYMEQSFQSLNPVLSLHINPAQVEQARKNAAFTGTALEAWLVGGTWTAMDVCATGPTACPVMQTCSQTAWSSFSPDPKSELGAVKPDGRLR

SEQ ID NO: 7 - DNA, 2858 bp, Rat coronavirus
  CDS (56)..(2650): rat XT-2
gcgctcgcggccgggttgcagggccggacgcgcgccccgcgtcccgggcaggaagatggtggcgagcgcgcgggtgcagaagctggtgcggcgctacaagctggcgatcgccaccgcgctggccatcctgctgctgcagggcctggtggtgtggagcttcagcggcctggaggaggacgagccgggcgagaaaggaaggcagagaaagccacggccgctagaccctggagagggttccaaggacacggacagctctgcagggcgtaggggcagtgctgggagaaggcacggacggtggcggggccgcgccgagagcccaggtgtgcctgtggccaaggtggtgcgggcagtaaccagtcggcagagagccagccgacgggtgccccctgccccacctccagaggcaccaggccgccagaacctgagtggagcagcagccggggaagcactgataggtgccgccggcttcccacaacatggagacacagggagtgtggagggtgcccctcagcccacggataacagcttcactccgaagtgtgagattgtgggcaaggatgcactgtcagcactggcccgggccagcaccaagcattgtcaacaagagattgctaatgtagtgtgcctgcaccaagctgggaacctaatgcccaagtctgtgccccggcactgccagctggctggcaaggtgagccctggcatccagtgggaagaggtccgggcccagcagcctgtgagtggccctctggtacgcatcgcctacatgctggtggttcacggccgtgctgtgcgccagctgaagcgtcttctgaaggccgtctaccacgaggagcacttcttttatattcatgtggacaagcgttccaactacctgtaccgggaggtggtagagctggcccagcactacgacaatgtacgggtaacaccttggcgcatggtcaccatctggggtggggctagccttctgaggatgtacctgcggagcatgaaggaccttctggagactcctggctggacctgggacttcttcatcaacctgagcgctactgactatccaaccaggacgaatgaggagctggtagcgttcttatccaagaaccgggacaagaatttcctcaagtcacacgggcgagacaattccaggttcatcaagaaacaaggcctggaccggcttttccatgagtgtgattcccacatgtggcgcctgggtgaacggcagatcccggcaggcatcgtggtggatggtggctctgactggttcgtgctgacacgcagctttgtggaatatgtggtgtatacagaggatcctctggtggcccagcttcgccagttctatacatacacattgcttccagccgagtccttcttccacacagtgctggagaacagcccagcctgtgagagcctagtggacaacaacctgcgggttaccaactggaaccggaagctgggctgcaagtgccagtacaagcacatcgtggactggtgtggctgctcccccaacgacttcaagccacaggacttcctgcggcttcagcaagtctccagacccaccttctttgcccggaagtttgagtcgactgtgaaccaggaagtcctggaaattttggacttccacctgtatggcagctacccacccggcaccccagccctcaaggcctactgggagaacatctacgacatggccgatggccctagtggactcagcgatgtcctactcactgcttacacagcctttgcccgtatcagtctgcgtcatgctgccactgtttccccactggccactgcagtctgcaggtttgagcccagggggttgccgtccagcgtgcacctgtatttctatgacgaccatttccagggctacctggtgacgcaggcagtgcagccctcagcccaggggccagcagagacacttgagatgtggctgatgccccagaggttgctgaagccgttggggcacagtgaccaggccagccggctccagagtctggaggttggcactgagtgggaccccaaagaacgtctcttccggaactttgggggcctgttgggaccactggatgaacctgtggccatgcagcgctgggcccggggccccaacctcacagccactgtggtctggattgaccccacctatgttgtggccacatcctatgacatcacggtagatgcggacactgaagtcacgcagtacaagcccccactgagcctgccactgcggccaggagcctggactgttcgattgcttcagttctgggagcccctgggtgagacccgcttcctcgtgctgccattgaccttcaaccacaaactacctctcaggaaagatgatgccagctggctgcatgcgggaccaccccacaacgaatacatggaacagagtttccagggactaagtggcatcctgaatctgcctcagccagaggccgtggaggaggctgcccggcggcacacagagctcacaggtcctgcacttgaggcctggacagatggggaactgagcagtttctggtctgttgcaggattgtgtgccatagggccttcttcttgtccctccctggagctctgcagactgaccagctggagctctctgtctcctgaccccaagtcagagctggggcctgtcaaagctgacggacgactcaggtagcaggaccccagccagcacaacccggaggagccggggaattgcaccttacagacaatggagggacgtctctcctctggtagccagggagccctagaacagtggtggggctgtctgtctgtcaggctttcctaggaaatggacgacatcaggccctgctgagatccatgaagtttccccactggaggagggagccccactggcagcacag

SEQ ID NO: 8 - protein, 864 aa, Rat coronavirus
MVASARVQKLVRRYKLAIATALAILLLQGLVVWSFSGLEEDEPGEKGRQRKPRPLDPGEGSKDTDSSAGRRGSAGRRHGRWRGRAESPGVPVAKVVRAVTSRQRASRRVPPAPPPEAPGRQNLSGAAAGEALIGAAGFPQHGDTGSVEGAPQPTDNSFTPKCEIVGKDALSALARASTKHCQQEIANVVCLHQAGNLMPKSVPRHCQLAGKVSPGIQWEEVRAQQPVSGPLVRIAYMLVVHGRAVRQLKRLLKAVYHEEHFFYIHVDKRSNYLYREVVELAQHYDNVRVTPWRMVTIWGGASLLRMYLRSMKDLLETPGWTWDFFINLSATDYPTRTNEELVAFLSKNRDKNFLKSHGRDNSRFIKKQGLDRLFHECDSHMWRLGERQIPAGIVVDGGSDWFVLTRSFVEYVVYTEDPLVAQLRQFYTYTLLPAESFFHTVLENSPACESLVDNNLRVTNWNRKLGCKCQYKHIVDWCGCSPNDFKPQDFLRLQQVSRPTFFARKFESTVNQEVLEILDFHLYGSYPPGTPALKAYWENIYDMADGPSGLSDVLLTAYTAFARISLRHAATVSPLATAVCRFEPRGLPSSVHLYFYDDHFQGYLVTQAVQPSAQGPAETLEMWLMPQRLLKPLGHSDQASRLQSLEVGTEWDPKERLFRNFGGLLGPLDEPVAMQRWARGPNLTATVVWIDPTYVVATSYDITVDADTEVTQYKPPLSLPLRPGAWTVRLLQFWEPLGETRFLVLPLTFNHKLPLRKDDASWLHAGPPHNEYMEQSFQGLSGILNLPQPEAVEEAARRHTELTGPALEAWTDGELSSFWSVAGLCAIGPSSCPSLELCRLTSWSSLSPDPKSELGPVKADGRLR

SEQ ID NO: 9 - protein, 17 aa, Artificial Sequence
  Description of Artificial Sequence peptide
CSRQKELLKRKLEQQEK

SEQ ID NO: 10 - protein, 11 aa, Artificial Sequence
  Description of Artificial Sequence peptide
QEEEGSGGGQK

SEQ ID NO: 11 - protein, 12 aa, Artificial Sequence
  Description of Artificial Sequence peptide
SENEGSGMAEQK

SEQ ID NO: 12 - protein, 12 aa, Artificial Sequence
  Description of Artificial Sequence peptide
TENEGSGLTNIK

SEQ ID NO: 13 - DNA, 28 bp, Artificial Sequence
  Description of Artificial Sequence  primer
gtgggtatgcagaagtgggggaagggac

SEQ ID NO: 14 - DNA, 18 bp, Artificial Sequence
  Description of Artificial Sequence primer
ccctccgcaatgcctaca

SEQ ID NO: 15 - DNA, 20 bp, Artificial Sequence
  Description of Artificial Sequence primer
actgaggtcacgcaatacaa

SEQ ID NO: 16 - DNA, 25 bp, Artificial Sequence
  Description of Artificial Sequence primer
gccgcactcaggttgatgaagaagt

SEQ ID NO: 17 - DNA, 25 bp, Artificial Sequence
  Description of Artificial Sequence primer
accaccaggacaaaggcgattctga

SEQ ID NO: 18 - DNA, 20 bp, Artificial Sequence
  Description of Artificial Sequence primer
agtcgaacagtccagggccc

SEQ ID NO: 19 - DNA, 20 bp, Artificial Sequence
  Description of Artificial Sequence primer
cacgatctcgcacttggggg

SEQ ID NO: 20 - DNA, 32 bp, Artificial Sequence
  Description of Artificial Sequence primer
ctattcgatgatgaagataccccaccaaaccc

SEQ ID NO: 21 - DNA, 20 bp, Artificial Sequence
  Description of Artificial Sequence primer
ggaagagctgggtgtggaat

SEQ ID NO: 22 - DNA, 20 bp, Artificial Sequence
  Description of Artificial Sequence primer
tttcccgttgagatcctgct

SEQ ID NO: 23 - DNA, 20 bp, Artificial Sequence
  Description of Artificial Sequence primer
acagacagcaacaacgagaa

SEQ ID NO: 24 - DNA, 19 bp, Artificial Sequence
  Description of Artificial Sequence primer
aaaggaaggcagaggaagc

SEQ ID NO: 25 - DNA, 20 bp, Artificial Sequence
  Description of Artificial Sequence primer
acccctccactgtctgtaag

SEQ ID NO: 26 - protein, 5 aa, Artificial Sequence
  Description of Artificial Sequence digestion peptide
ELGAK

SEQ ID NO: 27 - protein, 4 aa, Artificial Sequence
  Description of Artificial Sequence digestion peptide
ELLK

SEQ ID NO: 28 - protein, 6 aa, Artificial Sequence
  Description of Artificial Sequence digestion peptide
DMNFLK

SEQ ID NO: 29 - protein, 10 aa, Artificial Sequence
  Description of Artificial Sequence digestion peptide
IASPPSDFGR

SEQ ID NO: 30 - protein, 4 aa, Artificial Sequence
  Description of Artificial Sequence digestion peptide
LLLD

SEQ ID NO: 31 - protein, 13 aa, Artificial Sequence
  Description of Artificial Sequence digestion peptide
DFENVDNSNFAPR

SEQ ID NO: 32 - protein, 6 aa, Artificial Sequence
  Description of Artificial Sequence digestion peptide
PTFFAR

SEQ ID NO: 33 - protein, 12 aa, Artificial Sequence
  Unsure (Asp)...(Asp): Description of Artificial Sequence digestion peptide; Xaa = unidentified amino acid
LQFSEVGTDXDA

SEQ ID NO: 34 - protein, 11 aa, Artificial Sequence
  Description of Artificial Sequence digestion peptide
ELGAVKPDGRL

SEQ ID NO: 35 - protein, 12 aa, Artificial Sequence
  Description of Artificial Sequence digestion peptide
ELLKRKLEQQEK

SEQ ID NO: 36 - protein, 8 aa, Artificial Sequence
  Description of Artificial Sequence digestion peptide
LGLLMPEK

(19) United States
(12) Patent Application Publication
(10) Pub. No.: US 2005/0070001 A1
(43) Pub. Date: Mar. 31, 2005

(54) Xylosyltransferase and isoforms thereof

(76) Inventors: Knut Klessiek, Bad Oeynhausen (DE); Thomas Brinkmann, Bielefeld (DE); Christian Goetting, Loehne (DE); Joachim Kuhn, Lebach (DE)

Correspondence Address: LOCKE LIDDELL & SAPP LLP, 600 TRAVIS, 3400 CHASE TOWER, HOUSTON, TX, 77002-3095, US

(21) Appl. No.: 10/169,368

(22) PCT Filed: Dec. 28, 2000
(86) PCT No.: PCT/EP00/13311

(30) Foreign Application Priority Data
Dec. 30, 1999 (EP) 99126194.2
Dec. 1, 2000 (EP) 00126233.6

(51) Int. Cl. C12P 21/06; C07H 21/04; C12N 9/10

(52) U.S. Cl. 435/193; 435/69.1; 435/320.1; 435/325; 435/68.1; 536/23.2

(57) ABSTRACT

The invention relates to the isolation, purification and characterization of the enzyme xylosyltransferase (defined as “XT”). The invention describes for the first time that XT occurs in at least two isoforms (“XT-I”, “XT-II”). The invention relates furthermore to the recombinant cloning and expression of human and rat XT-I and XT-II and discloses their DNA and protein sequences. The enzymes according to the invention can be used as therapeutic agents and as diagnostic markers, e.g. for the determination of enhanced proteoglycan biosynthesis, and as biochemical markers for determination of several pathological processes such as systemic sclerosis.

The invention relates to the isolation, purification and characterization of the enzyme xylosyltransferase (defined as “XT”). The invention describes for the first time that XT occurs in at least two isoforms (“XT-I”, “XT-II”). The invention relates furthermore to the recombinant cloning and expression of human and rat XT-I and XT-II and discloses their DNA and protein sequences. The enzymes according to the invention can be used as therapeutic agents and as diagnostic markers, e.g. for the determination of enhanced proteoglycyn biosynthesis, and as biochemical markers for determination of several pathological processes such as systemic sclerosis.

### BACKGROUND OF THE INVENTION

Proteoglycans are polyanionic molecules widely expressed in animal cells and virtually every tissue. These abundant molecules are present in the extracellular matrix and on the cell surface and serve a wide range of functions. They are increasingly implicated as important regulators in many biological processes, such as extracellular matrix deposition, cell membrane signal transfer, morphogenesis, cell migration, normal and tumor cell growth and viral infection (Ruoslahti, 1989, J. Biol. Chem. 264, 13369-1372; Herold et al., 1994, J. Gen. Virol. 75, 1211-1222). Proteoglycans mediate diverse cellular processes through interaction with a variety of protein ligands. In most of these bindings electrostatic interactions with the glycosaminoglycan chains attached to the core protein are involved (Kjellen & Lindahl, 1991, Annu. Rev. Biochem. 60, 443-475). Thus, the biological activity of proteoglycans is intimately related to the glycosaminoglycan biosynthesis.

The sulfated glycosaminoglycans chondroitin sulfate, heparan sulfate, heparin and dermatan sulfate are bound to the proteoglycan core protein by a xylose-galactose-galactose binding region (Kjellen & Lindahl, 1991, l.c.). UDP-D-xylose:proteoglycan core protein β-D- [symbol] various species (Hoffmann et al., 1984, Connect. Tissue. Res. 12, 151-164), and it was shown that the enzyme is secreted from the endoplasmatic reticulum into the extracellular space together with chondroitin sulfate proteoglycans (Kähnert et al., 1991, Eur. J. Clin. Chem. Clin. Biochem. 29, 624-625; Götting et al., 1999, J. Invest. Dermatol. 112, 919-924). However, the processes resulting in the release of XT from the endoplasmatic reticulum or the Golgi compartments and the role of XT in the extracellular matrix are not yet known.

As XT is the initial step enzyme in the biosynthesis of the glycosaminoglycan linkage region and as it is secreted into the extracellular matrix to a great extent, XT activity was proposed to be a diagnostic marker for the determination of an enhanced proteoglycan biosynthesis and of tissue destruction (Weilke et al., 1997, Clin. Chem. 43, 45-51). XT activities in the synovial fluid were found to be significantly increased in chronic inflammatory joint diseases (Kleesiek et al., 1987, J. Clin. Chem. Clin. Biochem. 25, 473-481). Recent studies have shown that serum XT activity is a biochemical marker for the determination of fibrotic activity in systemic sclerosis (Götting et al.,1999, l.c.; Götting et al., 2000, Acta. Derm. Venereol. 80, 60-61.).

Up to now there was no success to isolate or characterize xylosyltransferase (XT), however some methods were described to measure and determine the activity of said enzyme from blood or body fluid samples of patients showing pathological effects such as scleroderma or chronic joint diseases (Stoolmiller, 1972, J. Biol. Chem. 247, 3525-3532). The samples were incubated with UDP-[¹⁴C]xylose and an appropiate acceptor. The incorporated radioactivity indicated the amount of XT activity. Acceptors used so far are proteoglycans, silk fibrin (Campbell et al., 1984, Anal. Biochem. 137, 505-516) and several peptides (Bourdon et al., 1987, Proc. Natl. Acad Sci. USA 84, 3194-3198). However, the all activity tests used herein did not allow precise determination of the lower XT activity in serum. A more specific acceptor protein is recombinant bikunin, the inhibitory component of human inter-α-trypsin inhibitor. Bikunin carries a single chondroitin, which is essential for the structure of the inhibitor. The chondroitin sulfate attachement site in the N-terminal region contains all elements responsible for recognition by XT. The complete recognition sequence is composed of the amino acids a-a-a-a-G-S-G-a-b-a, with a=E or D and b=G, E or D. This sequence was confirmed by determination of the Michaelis-Menten (Kₘ) constants for in vitro xylosylation of different synthetic proteins and peptides using an enriched XT preparation from conditioned cell culture supernatant of human chondrocytes. The constant was determined to be 22 μM, which was decreased 9-fold in comparison to deglycosylated core protein from bovine cartilage (188 μM) (Brinkmann et al., 1997, J. Biol. Chem. 272, 11171-11175). With recombinant bikunin as acceptor, a sensitive assay was developed that allows precise determination of XT activity in human serum and other body fluids (see: Weilke et al., 1997, l.c.). Using this assay an increased xylosyltransferase activity was determined in blood of patients with sclerotic processes of scleroderma, closely related to an elevated proteoglycan biosynthesis (Götting et al., 1999, l.c.).

The biosynthesis of glycosaminoglycans requires the coordinated action of a large number of glycosyltransferases. Isolation and cloning of these glycosyltransferases has been targeted for a long time, since the majority of these enzymes are only present in minute amounts. The structure and sequence of the glycosyltransferases involved in biosynthesis of the common glycosaminoglycan-protein linkage region has long remained unknown. Recent cDNA cloning of galactosyltransferase I (Okajima et al., 1999, J. Biol. Chem. 274, 22915-22918) and glucuruonyltransferase I (Kitagawa et al., 1998, J. Biol. Chem. 273, 6615-6618) identified 2 of the at least 4 enzymes involved in synthesis of the GlcAβ1-3Galβ1-3Galβ1-4Xylβ1-O-Ser structure.

Isolation, purification and characterization of XT involved in biosynthesis of the common carbohydrate-protein linkage structure has been hampered by difficulties in obtaining a sufficient amount of the source material. Since, as above mentioned, XT can be used as an additional biochemical marker for the determination of sclerotic activity in systemic sclerosis and some inflammatory disorders, it is a real need for an isolated, highly purified XT which can be produced by recombinant methods, which is necessary for diagnostic and therapeutic purposes. Moreover, the knowledge of the cDNA sequence of XT allows to use it on gene level such as in gene diagnostic or gene therapy.

Definitions

Above and below the term “XT” means xylosyltransferase (UDP-D-xylose:proteoglycan core protein β-D-xylosyltransferase (EC 2.4.2.26) deriving from any origin and includes all possible isoforms, if not otherwise pointed out. The term “hXT” has the meaning of human XT; the term “rXT” of rat XT. The terms “XT-I”, “XT-II”, “hXT-I”, “hXT-II”, “rXT-I2, “rXT-II” mean the specific isoforms 1 and 2 of XT according to the invention, wherein h and r have the indicated meanings.

Above and below the term “protein” means a protein, a protein fragment or a peptide, if not otherwise explained.

Above and below the term “recombinant protein” is defined as a protein which was produced by recombinant and biotechnological methods.

Above and below the term “XT” or “XT protein(s)” has the meaning of (a) protein(s) deriving from any source, if not otherwise stated out, having the biological activity and/or function of UDP-D-xylose:proteoglycan core protein β-D-xylosyltransferase.

Above and below the term “a protein having the biological activity of UDP-D-xylose:proteoglyean core protein β-D-xylosyltransferase” is defined as a protein which has XT—identical or XT—like functions and activities and comprises XT itself and possible mutations, variants, isoforms thereof, including insertions, deletions and substitutions of one ore more amino acids. The term includes also fragments or longer forms of XT as well as dimeric or multimeric forms thereof showing XT functions.

Above and below the term “isoform” means a second naturally occurring enzyme having the same biological activity as a first naturally occurring enzyme, however differing by another amino acid sequence.

### SUMMARY OF THE INVENTION

It was found that XT can be purified 4,700-fold with 1% yield from serum-free JAR choriocarcinoma cell culture supernatant. The isolation procedure includes a combination of ammonium sulfate precipitation, heparin affinity chromatography, ion exchange chromatography and protamine affinity chromatography. Amongst other proteins an unknown protein was detected by matrix-assisted laser desorption ionization mass spectrometry-time of flight analysis (MALDI-TOF) in the purified sample. The molecular weight of this isolated and purified XT protein was determined as 120.000 by SDS-polyacrylamide gel electrophoresis. The isolated protein was enzymatically cleaved by trypsin and endoproteinase Lys-C. Peptide fragments were sequenced by Edman degradation. Searches with the amino acid sequences in protein and EST databases showed no homology to known sequences. XT was enriched by immunoaffunity chromatography with an immobilized antibody against a synthetic peptide deduced from the sequenced peptide fragments and was specifically eluted with the antigen. In addition, XT was purified alternatively with an aprotinin affinity chromatography and was detected by western blot analysis in the enzyme-containing fraction.

Based on the partial amino acid sequence of their isolated and purified new enzyme (XT) derived from human JAR choriocarcinoma cell culture supernatant a novel cDNA was isolated according to the invention, encoding human XT-I enzyme using the degenerate reverse transcriptase-polymerase chain reaction method. The enzyme belongs to a novel family of glycosyltransferases having no homology to proteins of prior art 5′- and 3′-RACE were performed to isolate a novel cDNA fragment of 3726 bp with a single open reading frame encoding at least 827 amino acids with a molecular mass of 91.000. The human XT-I gene was located on chromosome 16p13.1 using radiation hybrid mapping, and extracts from CHO-K1 cells transfected with the XT-I cDNA in an expression vector exhibited marked XT activity. Furthermore, a new 3608-bp cDNA fragment encoding a novel protein of 865 amino acids was also isolated by PCR using degenerate primers based on the amino acid sequence of human XT-I. The amino acid sequence of this XT-II isoform displays 55% identity to the human XT-I. The XT-II gene is located on chromosome 17q21.3-17q22, and the exon/intron structure of the 15 kb gene was determined. RT-PCR analyses of XT-I and XT-II mRNA from various tissues confirmed that both XT-I and XT-II transcripts are ubiquitously expressed in the human tissues, although with different levels of transcription. Furthermore, the cDNAs encoding XT-I and XT-II from rat were cloned. The deduced amino acid sequences of rat xylosyltransferases displayed 94% identity to the corresponding human enzyme.

Therefore, it is an object of this invention to provide the following subject-matters:
An isoform of UDP-D-xylose:core protein β-D-xylosyZtransferase(XT);
a protein comprising a sequence of said isoform or a fragment thereof, having the biological activity of XT;
a corresponding protein deriving from human or rat sources (bXT, rXT);
a corresponding protein isolated from specific human tissue, wherein said hXT has a molecular weight of 120.000 under SDS PAGE conditions;
a corresponding recombinant protein, wherein said protein is hXT-I comprising at least 827 amino acids and having the amino acid sequence as depicted in FIG. 7B;
a corresponding isoform of hXT-I, termed as hXT-II, comprising 865 amino acids, exhibiting approximately 55% overall sequence identity to human hXT-I, and having, in more detail, the amino acid sequence as depicted in FIG. 8B.
a process for isolating and purifying a protein having the biological activity of human UDP-D-xylose:proteoglycan core protein β-D-xylosyltransferase (EC 2.4.2.26), said process is characterized by the following steps:
   (i) culturing from human tissue showing an enhanced XT activity, preferably from JAR choriocarcinoma cells (ATCC HTB-144), and harvesting the cell culture supernatant,
   (ii) fractionated ammonium sulfate precipitation of the supernatant of step (i),
   (iii) heparin affinity chromatography of the precipitate of step (ii),
   (iv) ion exchange chromatography of the step (iii) product, and
   (v) affinity chromatography of the step (iv) product, and optionally
   (vi a SDS-Polyacrylamide gel elektrophoresis of step (v);
recombinant forms of hXT and rXT, the corresponding DNA sequences (FIG. 7A, 8A, 9, 10) included as well as suitable expression vectors and expression host cell systems;
antibodies directed against any of the above or below mentioned XT proteins and their uses in immunological assays and diagnostic tools for determining said XT proteins;
pharmaceutical compositions comprising a XT protein as defined above and below, optionally together with a suitable pharmacologically acceptable carrier, diluent or excipient;
uses of said XT proteins for the manufacture of a medicament for the treatment of XT relevant diseases and disorders, wherein the xylosyltransferase enzymes according to the invention can either be used directly as therapeutic drug in pathological situations where a deficiency of said enzyme and its isoforms can be detected;
uses of said XT enzymes for the manufacture of a medicament for the treatment of diseases and disorders which are caused or accompanied by increased levels of said enzymes, e.g. in sclerotic diseases and chronic joint diseases, wherein said medicament is an inhibitor or antagonist of said XT proteins;
uses of said XI proteins as diagnostic markers for the diagnosis of above- and below-mentioned diseases and pathological symptoms and uses of said DNA molecules as gene markers;
methods of screening for compounds which are capable to inhibit the activity of said XT proteins according to the invention using known methods for determining the activity of XT.

### DETAILED DESCRIPTION

General

UDP-D-xylose: proteoglycan core protein β-D-xylosyltransferase can be isolated and purified from JAR choriocarcinoma cell culture. The isolated protein according to the invention is a single stranded polypeptide with a molecular weight of 120.000. The protein was enzymatically cleaved and eleven peptide fragments were sequenced by Edman degradation (see Examples). XT is present only in very small amounts in animal tissues but unlike other glycosyltransferases, more than 90% of XT is enriched in the medium of cultured cells. The highest secretion of XT activity was measured in JAR choriocarcinoma cell culture, in which sternal cartilage chondrocytes and 21 different human cell lines were examined. To produce a highly enriched XT solution for the isolation of XT, JAR choriocarcinoma cells were adapted to hollow fiber culture conditions using a novel bioreactor (TECNOMOUSE) and Ultradoma-PF medium without serum addition as nutrient. For purification of XT a combination of classic separation methods and new affinity matrices was employed. Therefore, a heparin matrix was used as an affinity ligand for the XT. When applied to immobilized heparin, XT was completely adsorbed at the matrix and the XT activity was eluted only with a high salt concentration after most contaminating proteins have been removed from the matrix. Protamine chloride is well-known as cationic activator for several sulfotransferases, so the effect of protamine chloride on the XT was investigated. An increased XT activity was measured when protamine chloride was added to the XT assay solution (see Examples), indicating an interaction of these arginine rich proteins with XT. Therefore, a protamine chloride affinity matrix was synthesized using an aldehyde activated perfusion medium as support. The interaction of XT with this affinity matrix resulted in a 13.6 times enrichment of XT. The protamine chloride affinity chromatography was the most efficient purification step during the isolation of the XT. Immobilized aprotinin, a Kunitz-type proteinase inhibitor, was found to be another appropriate affinity matrix for enrichment of XT, as it was able to adsorb XT quantitatively. Therefore, it was used for alternative purification of XT. Different lines of evidence showed that the isolated protein corresponds to the XT: (a) XT activity was enriched using immobilized antibodies raised against a synthetic peptide deduced from the 120.000 protein, and the XT activity could be competitively eluted with this peptide. (b) Immunoblot analysis of aprotinin affinity purified XT corresponds with the 120.000 protein. However, non-reducing and non-denaturating gel filtration chromatography with heparin affinity-purified XT from JAR cell culture supernatant shows an additional peak of XT activity at a molecular weight of approximately 500.000. XT is associated with proteoglycans. Treatment of XT with N-glycosidase F resulted in a decrease of the molecular weight from 120.000 to 116.000, suggesting that the XT is a glycoprotein. A comparison of the molecular mass of XT with other glycosyltransferases involved in biosynthesis of proteoglycans shows that the XT is larger than the other enzymes. Another difference is that nearly all proteoglycan glycosyltransferases are tightly bound to the membrane of the endoplasmic reticulum, whereas XT is secreted into the extraceuular space. XT according to the invention contains like many glycosyltransferases a D×D motif, suggesting that this motif is involved in binding the metal-ion cofactor and the donator substrate (Gastinel et al., 1999, EMBO J. 18, 3546-3557). A D×D sequence was also found in peptide 8 obtained from the enzymatically cleaved XT.

The invention discloses for the first time the molecular cloning and expression of human and rat XT. Surprisingly, XT is found in at least two isomeric forms, which are termed according to the invention as XT-I and XT-II. Based on the amino acid sequence a novel cDNA was cloned, which encodes a protein of at least 827 amino acids with a molecular mass of 91.000. A D×D motif was identified in the XT-I amino acid sequence using hydrophobic cluster analysis. This motif has been observed in many glycosyltransferases, and is involved in the coordination of a divalent cation in the binding of the nucleotide-sugar (Breton & Imberty, 1999, Curr. Opin. Struct. Biol. 9, 563-571.). The translation initiation codon of the XT-I cDNA could not be yet cloned probably due to strong secondary structures in the 5′-region of the XT-I mRNA. The XT-I protein isolated from human JAR choriocarcinoma cell culture supernatant migrated on SDS-PAGE with a molecular mass of 120.000 and the protein size could be further reduced after N-glycanase digestion. These findings indicate that the cloned cDNA represents at least 90% of the coding region of human XT-I.

Another cDNA was completely identified from human placenta RNA, which was similar but not identical to the hXT-I cDNA. The new cDNA encodes a protein of 865 amino acids with a molecular weight of 97.000. This novel protein termed XT-I has a proline-rich region located near the amino-terminus and the type II transmembrane topology characteristic of many other glycosyltmnsferases cloned to date. The hXT-II protein was similar to the human XT-I with an overall sequence identity of 55%. The similarity of the predicted amino acid sequences was low (<10%) at the amino-terminal end. However, large stretches at the C-terminal region, where the catalytic domain was found to be located in glycosyltransferases, are very conserved with an identity of more than 80% in both proteins. These findings let conclude that the XT-II gene encodes another human xylosyltransferase, although the catalytic activity and the biological role of XT-II remain to be elucidated in detail.

Since alterations in XT activity have been reported to be associated with fibrotic and sclerotic alterations of connective tissue (Götting et al., 1999; Götting et al., 2000, l.c.), the present findings provide molecular tools to study the function and the regulated expression of human XT as well as the molecular mechanisms of these diseases.

Production of XT

JAR choriocarcinoma cells which had been adapted to growth in the serum-free Ultradoma-PF medium secreted XT activity into the cell culture supernatant. During the exponential growth XT activity and total protein concentration in the supernatant of a traditional cell culture system (T-flasks) were determined as 0.2 mU/l and 0.1 g/l, respectively. The cells were cultivated using three “Tecnomouse”® bioreactor systems. Each bioreactor contained five culture casettes. About 10⁷ cells per culture casette were inoculated and medium probes of about 0.5 ml were taken every day to determine the viability of the cells and the glucose, and lactate concentration as well as the XT activity of the cell culture supernatant. The cells amounted in the probes varies from 10⁵ to 10⁷ cells per ml with viability between 40 and 70%. XT production increased within three weeks after cell inoculation, reaching a plateau of approximately 30 mU/l. After three months the culture cassette was removed. Harvesting was carried out every two days, collecting 10 ml cell culture supernatant per culture cassette. The cell-free supernatant was collected and stored at −75° C. Mean XT activity in the supernatant of the high density culture was determined as 29.0 mU/l, while the total protein concentration was estimated as 4.8 g/l. In total 18.5 l high density cell culture supernatant was collected and yielded 535.8 mU XT.

Isolation and Purification of XT from Cell Culture Supernatant

XT was purified form 18.5 l supernatant (equivalent to 2.000 l normal cell culture supernatant) of serum-free cultivated JAR choriocarcinoma cells to an apparent homogeneity of about 4700-fold purification. A summary of the purification steps for isolation of XT is shown in Table 1. XT activity of the crude supernatant was approximately 0.006 mU/mg protein. The purification method according to the invention comprises four, preferably five different steps. It is possible, that one step can be achieved more than once if necessary.

Step 1: Fractionated ammonium sulfate precipitation XT of the ammonium sulfate precipitable fraction was dissolved in 0.46 l buffer A with solubilization of 79.5% of the original activity.

Step 2: Heparin affinity chromatography on POROS 20 HE 4 ml of XT-enriched solution from step 1 was loaded onto the POROS 20 HE column. XT activity was completely retained on the column. More than 70% of total protein passed through the column. Contaminating protein was eluted at a low NaCl concentration. 44% of the XT activity bound to the heparin matrix emerged at 0.5 M NaCl (FIG. 1A).

Step 3: Ion exchange chromatography on POROS 20 HQ 4 ml of the desalted XT-containihg fraction from step 2 was loaded onto the POROS 20 HQ column equilibrated in buffer A. More than 98% of the XT activity bound to the resin. The column was then eluted stepwise with NaCl in buffer A (FIG. 1B). XT-containing fractions were collected.

Step 4: Affinity chromatography on protamine chloride The product of step 3 was desalted and concentrated using dia- and ultrafiltration. 100 μl of the protein solution was applied to the POROS protamine chloride column previously equilibrated with buffer A. Approximately 95% of the transferase activity bound to the column, whereas 75% of the contaminating protein did not. Additional proteins were eluted with buffer A containing low NaCl concentrations. Enzyme activity was eluted at approximately 0.15 M NaCl (FIG. 1C). The enzyme activity was stable for at least 6 months at −75° C.

Step 5: SDS-Polyacrylamide gel electrophoresis (SDS-PAGE) XT-containing fractions from step 1-4 were subjected to SDS-PAGE on a 4-12% gradient polyacrylamide gel (FIG. 2, panel A). Coomassie-stained protein bands were excised and characterized by MALDI-TOF mass spectrometry after tryptic digestion. The molecular weight of an unknown protein was determined as 120.000 (FIG. 2, panel B). Step 5 is an optionally step.

TABLE 1
Summary of single purification steps employed for isolation of XT from
18.5 l of high density JAR cell culture supernatant.

| | | Total | Total | Spec. | Purifi- | Recov- |
|---|---|---|---|---|---|---|
| | Volume | activity | protein | activity | cation- | ery |
| Step | ml | 10-3 × units | mg | 10-3 × units/mg | fold | % |
| JAR high density | 18,500 | 535.8 | 89,355.0 | 0.006 | 1 | 100 |
| cell culture | | | | | | |
| Supematant | | | | | | |
| Ammonium | 460 | 426.0 | 8,937.8 | 0.048 | 8 | 79 |
| sulfate | | | | | | |
| precipitation | | | | | | |
| Heparin | 50 | 108.3 | 473.0 | 0.229 | 40 | 35 |
| affinity | | | | | | |
| chromatography | | | | | | |
| Ion exchange | 5 | 91.0 | 43.1 | 2.090 | 348 | 17 |
| Chromatography | | | | | | |
| Protamine | 1 | 6.83 | 0.24 | 28.458 | 4,743 | 1 |
| affinity | | | | | | |
| chromatography | | | | | | |

Amino acid sequence analysis of XT. The MW 120.000 protein from the excised band was digested with trypsin and endoproteinase Lys-C. The proteolytic fragments were separated by reversed-phase HPLC, and selected peptides were subjected to automatic amino acid sequence analysis. Table II shows the obtained 11 amino acid sequences determined by Edman degradation and mass spectrometry.

TABLE II
X represent a not identified residue. The masses of three peptides
were observed in the MALDI mass spectrum of the enzymatically digested
MW 120.000 protein. Calculated mass values were obtained from the
sequence obtained.

| | observed mass | calculated mass |
|---|---|---|
| Peptide | (M + H⁺) | (M + H⁺) |
| (1) E L G A K | | |
| (2) E L L K | | |
| (3) D M N F L K | | |
| (4) I A S P P S D F G R | 1045.5 | 1045.5 |
| (5) L L L D | | |
| (6) D F E N V D N S N F A P R | 1524.7 | 1525.7 |
| (7) P T F F A R | | |
| (8) L Q F S E V G T D X D A | | |
| (9) E L G A V K P D G R L | 1152.6 | 1153.7 |
| (10) E L L K R K L E Q Q E K | | |
| (11) L G L L M P E K | | |

Immunochemical Detection of XT

Polyclonal antibodies against the synthetic peptide CSRQKELLKRKLEQQEK deduced from the peptides 2 and 10 of the enzymatically cleaved MW 120.000 protein were covalently bound on POROS 20 PA. About 50% of the XT activity of an applied sample was bound (FIG. 3, panel A) when a partially purified XT sample obtained by heparin affinity chromatography purification step 2) was loaded onto the column. 58% of the adsorbed XT activity was eluted with 150 mM NaCl, and the rest was eluted with 12 mM HCl. Furthermore, the adsorbed XT activity was also eluted from the solid phase when 100 μl (1 mg/ml) of the synthetic peptide was added to the mobile phase (FIG. 3, panel C). When immobilized preimmune serum was used as affinity matrix (negative control) no XT activity was adsorbed to or eluted from the matrix (FIG. 3, panel B). The desalted XT fraction after heparin affinity chromatography (purification step 2) was loaded on an aprotinin affinity column. The elution profile shows four major protein peaks (FIG. 4, panel I). 61% of the XT activity adsorbed to the aprotinin matrix emerged at 0.30 M NaCl and another 21% at 0.54 M NaCl. A single MW 120.000 band of the XT-containing fractions was detected by western blot analysis with the polyclonal antibodies (FIG. 4, panel B).

Determination of the Molecular Weight of XT

100 μl of heparin affinity purified XT was separated under non-reducing and non-denaturating conditions using a TSK G3000 SW column. Two XT activity maxima were detected at MW 500.000 and 120.000 (FIG. 5, panel A). The molecular mass of the MW 120.000 protein was reduced about 3% after N-glycosidase F digestion as shown by SDS-PAGE (FIG. 5, panel B).

PCR-Based Cloning of Human XT-I cDNA

Based upon the amino acid sequence of 4 peptides degenerate primers were designed for cloning the XT-I cDNA (FIG. 6). When primers SPPS1 and Lysc-inv were used in a PCR with the first strand cDNA of SW1353 chondrosarcoma poly(A)⁺ mRNA as template, a major band of approximately 690 bp was observed. After subcloning and sequencing a previously unknown DNA sequence was obtained from clone pCG114-29. PCR amplification with the primers DF1 and a mixture of Inv2b and Inv2c resulted in a 1724 bp fragment, which was subcloned and sequenced. The deduced amino acid sequence of the clone pCG111-4 was identical to 6 of the sequenced peptides from human XT-I. The cloning strategy of rapid amplification of cDNA ends (RACE) (Chenchik et al., 1996, BioTechniques 21, 526-534) was employed to clone the complete coding sequence of the XT-I cDNA. The largest DNA fragment obtained from the 3′-RACE reaction was 1.6 kb and consisted of a 3′-untaanslated region of 1240 bp. The 5′ RACE reaction was performed with different primers derived from the nucleotide sequence of XT-I cDNA, but the 5′-untranslated region of human XT-I cDNA could not be cloned using this method. All DNA fragments obtained contained just the known cDNA sequence and an additional 80 bp of coding sequence. Thus, a PCR-based screening approach using cDNA libraries as template was employed for cloning of the 5′-untranslated region. All DNA fragments obtained from the screening of 3 different human cDNA libraries stopped at the same nucleotide, indicating that stable secondary structures of the XT-I mRNA prevent the synthesis of cDNA of the 5′ untranslated region during the reverse transcription reaction. However, the translation initiation codon of human XT-I could not be yet cloned. The combined cDNA of human XT-I contained 3726 bp (FIG. 7A) with a single open reading frame encoding at least 827 amino acids with a molecular mass of a least 91.000. The deduced amino acid sequence contained 3 potential N-glycosylation sites (FIG. 7B). Analysis of the amino acid sequence using the hydrophobic cluster analysis (Gaboriaud et al., 1987, ) revealed the presence of a common D×D motif at position 182, which has been shown to be essential for binding nucleotide-sugars in glycosyltransferases .

PCR-Based Cloning of Human XT-II Isoform cDNA

The degenerate primers PFF-sense and Lysc-inv1 which were designed upon the amino acid sequence of proteolytic cleaved peptides of human XT-I were used in a PCR reaction with first strand cDNA of placenta poly(A)⁺ mRNA as template. The PCR amplification resulted in a minor band of 1.1 kb, which was cloned and sequenced. The determined nucleotide sequence was similar but not identical to the XT-I cDNA sequence, indicating that the fragment encodes a XT-II isoform. To clone the complete coding sequence of the novel cDNA, the RACE strategy was employed. The cDNA finally obtained was 3608 bp (FIG. 8A) and contained a single open reading frame encoding a protein of 865 amino acids with a molecular mass of 97.000. The 3′-untranslated region is 850 bp and a 5′-untranslated region of 149 bp was identified with an in-frame stop codon upstream of the ATG codon. A Kyte-Doolittle hydropathy analysis (Kyte & Doolittle, 1982, J. Mol. Biol. 157, 105-132) of the deduced amino acid sequence revealed one potential membrane-spanning region consisting of 16 hydrophobic amino acid residues at position 16 to 32, which appears to result in a type II transmembrane CHO-K1 cells and absorbed from the medium by immunoprecipitation with anti-Xpress-antibodies and protein G agarose beads to eliminate endogenous XT activity. The enzyme-bound beads were used as an enzyme source and assayed for XT activity as shown in Table 3 (see below). No detectable XT activity was recovered by the affinity purification from a control transfection sample. The substrate specificity of the recombinant XT-I was similar to that of the XT-I isolated from human body fluids and cell culture supernatants. The recovered enzyme activity of the recombinantly expressed XT-I could be completely inhibited by addition of 250 U of heparin. As specific inhibition of human XT activity by heparin has been demonstrated previously (Kleesiek et al., 1987, l.c.), these results clearly indicate that the expressed protein is the human XT. However, no enzymatic transfer of xylose to the acceptor peptides used in this study was observed when expressing XT-II fused to the aminoterminal peptide tag in CHO-K1 cells.

To identify the XT-I reaction products, the bikunin peptide was labeled with [¹⁴C]-D-xylose using the XT-I-bound beads as an enzyme source. The products were subsequently subjected to the linkage-specific digestion of the bound [¹⁴C]-D-xylose with α- and β-xylosidase and alkaline β-elimination. Incubation of the reaction products with β-xylosidase resulted in the release of 74% of the incorporated [¹⁴C]xylose, whereas only less than 4% of the peptide-bound xylose was digested after treatment with α-xylosidase. The alkaline cleavage of the O-glycosidic linkage between the xylose and the β-hydroxyamino acid serine in the presence of borohydride lead to the liberation of more than 97% of the enzymatically transferred [¹⁴C]xylose. In all the experiments performed no significant amount of [¹⁴C]-D-xylose was incorporated without the addition of the bikurin peptide as acceptor. These results clearly indicate that a xylose residue was transferred to the hydroxyamino acid serine of the bikunin peptide through a β-linkage. In conclusion the expressed protein was identified as UDP-D-xylose:proteoglycan core protein β-D-xylosyltransferase (EC 2.4.2.26).

TABLE 3
Xylosyltransferase activity of recombinant XT-I expressed in CHO-K1
cells. XT activity of the enzyme fractions using different acceptors
and the inhibition of enzyme activity by addition of heparin is shown.
The synthetic bikunin, L-APP and L-APLP2 homologous peptides have
been previously proved to be good acceptors for XT mediated xylosylation
(Brinkmann et al., 1997; Götting et al., 1998). No XT activity was
detected in samples from mock-transfected cells after affinity
purification. n.d., not detected (detection limit, 20 μU/l).

| | XT Activitiy | |
|---|---|---|
| Acceptor | pCG227-XT | +heparin |
| Recombinant bikunin | 2854 | n.d. |
| Bikunin peptide QEEGSGGGGQK | 463 | n.d. |
| L-APLP2 peptide SENEGSGMAEQK | 515 | n.d. |
| L-APP peptide TENEGSGLTNIK | 492 | n.d. |
| Peptide SGG | n.d. | n.d. |
| Chondroitin sulfate A | n.d. | n.d. |
| Chondroitin sulfate C | n.d. | n.d. |

Tissue-Specific Expression of XT-I and the XT-II Isoform

The expression of XT-I and XT-II isoform gene was examined in various human tissues using a RT-PCR based method with normalized first-strand cDNA. Each PCR yielded a single product with predicted nucleotide lengths of 490 bp for XT-I and 717 bp for XT-II, although the amount of the amplified product varied (FIG. 11). Amplification of XT-I and XT-II fragments resulted in a product visible by ethidium bromide staining after 36 cycles, whereas the DNA fragment corresponding to the abundant glyceraldehyde-3-phosphate dehydrogenase transcript was visible after 20 cycles. The greatest abundance of XT-I expression was detected in the placenta, kidney and pancreas and only a very weak expression was detected in skeletal muscle. The greatest abundance of XT-II isoform is expressed in the kidney and pancreas.

Cellular Distribution of XT Activity in Cultured CHO-K1 Cells

After an incubation period of 3 days cultured CHO-K1 cells were harvested and the XT activity was determined in the spent culture supernatant and the cell lysates. 92% of the total XT activity was found to be located in the cell culture medium (93.1 mU/10⁶ cells, SD 9.58), whereas only 2% was detected in the cell lysates (2.03 mU/10⁶ cells, SD 0.44). 6% of the total XT activity (5.82 mU/10⁶ cells, SD 2.18) was released from the membrane-containing fractions after addition of the detergence Triton X-100 indicating that less than 1/10th of XT is bound to cellular membranes.

Pharmaceutical and Diagnostic Use

The xylosyltransferase enzymes according to the invention can be used directly as therapeutic drug in pathological situations where a deficiency of said enzyme and its isoforms can be detected. As pointed out above XT enzyme may be overexpressed in some diseases and disorders. In these cases inhibitors and/or antagonists of XT-I and or XT-II can be used. Thus, the invention relates also to the use for the manufacture of a medicament for the treatment of diseases which are caused by increased levels of said enzymes, wherein said medicament is an inhibitor or antagonist of xylosyltransferase.

As mentioned above the protein according to this invention can be used as diagnostic means to evaluate pathological conditions. As used herein, the term “pharmaceutically acceptable carrier” means an inert, non toxic solid or liquid filler, diluent or encapsulating material, not reacting adversely with the active compound or with the patient. Suitable, preferably liquid carriers, are well known in the art. The formulations according to the invention may be administered as unit doses containing conventional non-toxic pharmaceutically acceptable carriers, diluents, adjuvants and vehicles which are typical for parenteral administration.

The term “parenteral” includes herein subcutaneous, intravenous, intra-articular and intratracheal injection and infusion techniques. Also other administrations such as oral administration and topical application are suitable. Parenteral compositions and combinations are most preferably adminstered intravenously either in a bolus form or as a constant fusion according to known procedures. Tablets and capsules for oral administration contain conventional excipients such as binding agents, fillers, diluents, tableting agents, lubricants, disintegrants, and wetting agents. The tablets may be coated according to methods well known in the art.

Unit doses according to the invention may contain daily required amounts of the protein according to the invention, or sub-multiples thereof to make up the desired dose. The optimum therapeutically acceptable dosage and dose rate for a given patient (mammals, including humans) depends on a variety of factors, such as the activity of the specific active material employed, the age, body weight, general health, sex, diet, time and route of administration, rate of clearance, enzyme activity (units/mg protein), the object of the treatment, i. e., therapy or prophylaxis and the nature of the disease to be treated.

Therefore, in compositions and combinations in a treated patient (in vivo) a pharmaceutical effective daily dose of the protein of this invention (hXT, hXT-I, hXT-II) is between about 0.01 and 100 mg/kg body weight (based on a specific activity of 100 kU/mg), preferably between 0.1 and 10 mg/kg body weight. According to the application form one single dose may contain between 0.5 and 10 mg of hXT.

### SHORT DESCRIPTION OF THE FIGURES

FIG. 1: Purification of XT

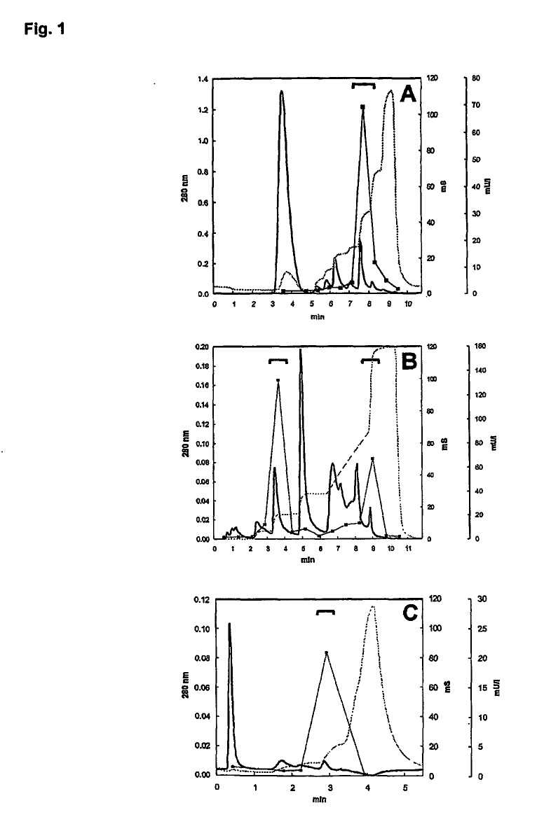

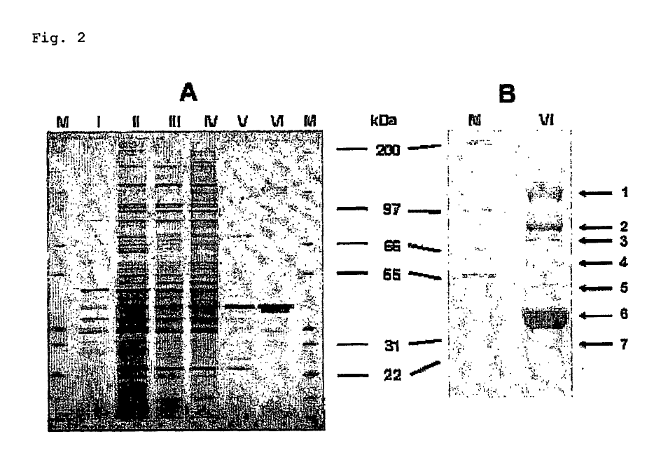

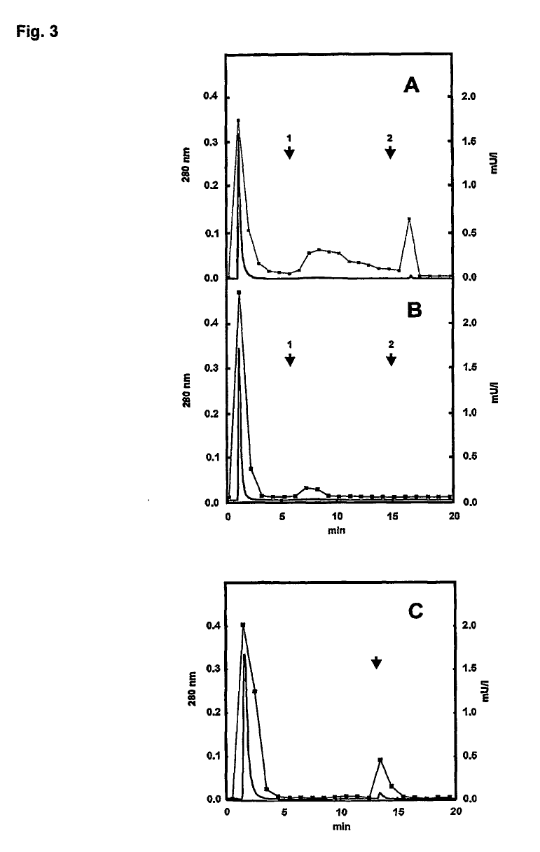

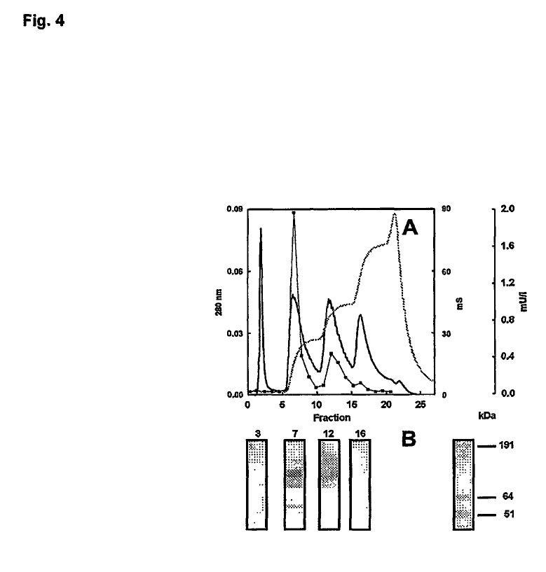

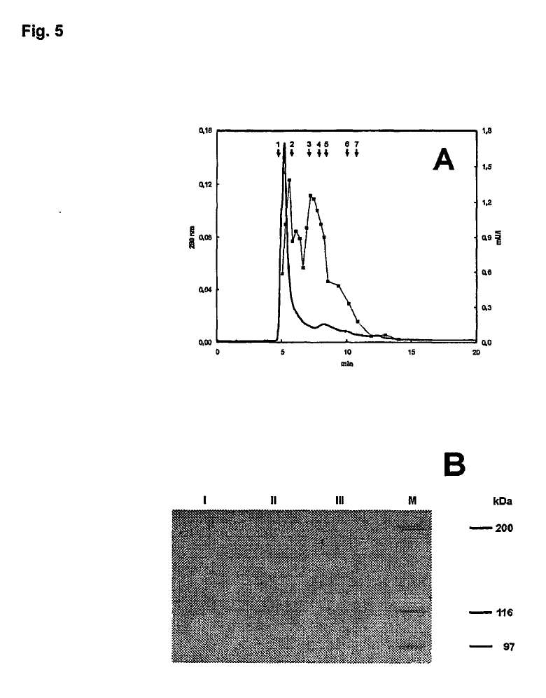

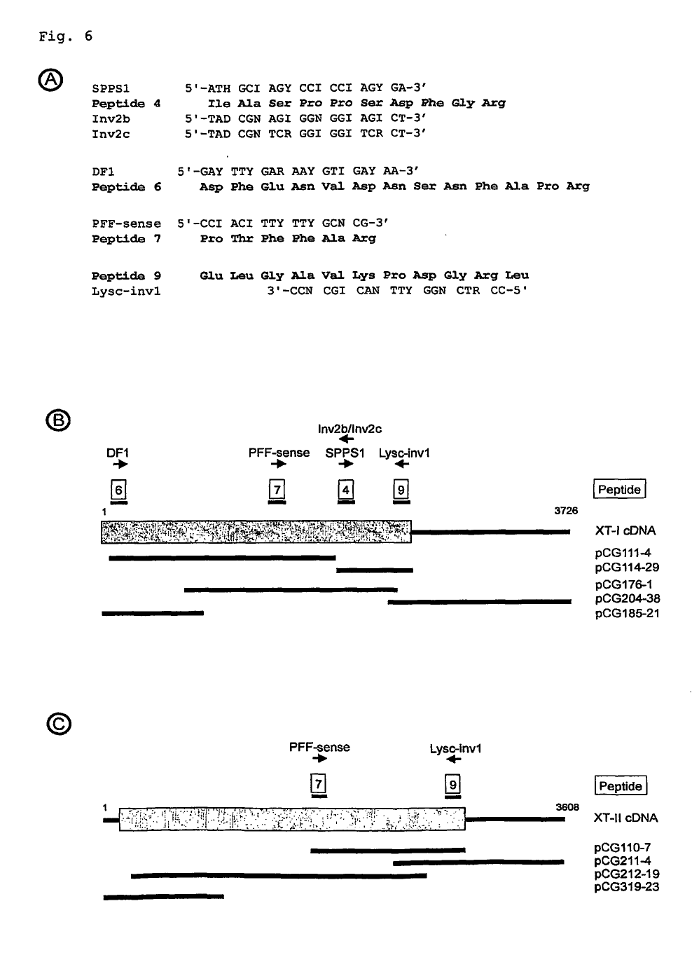

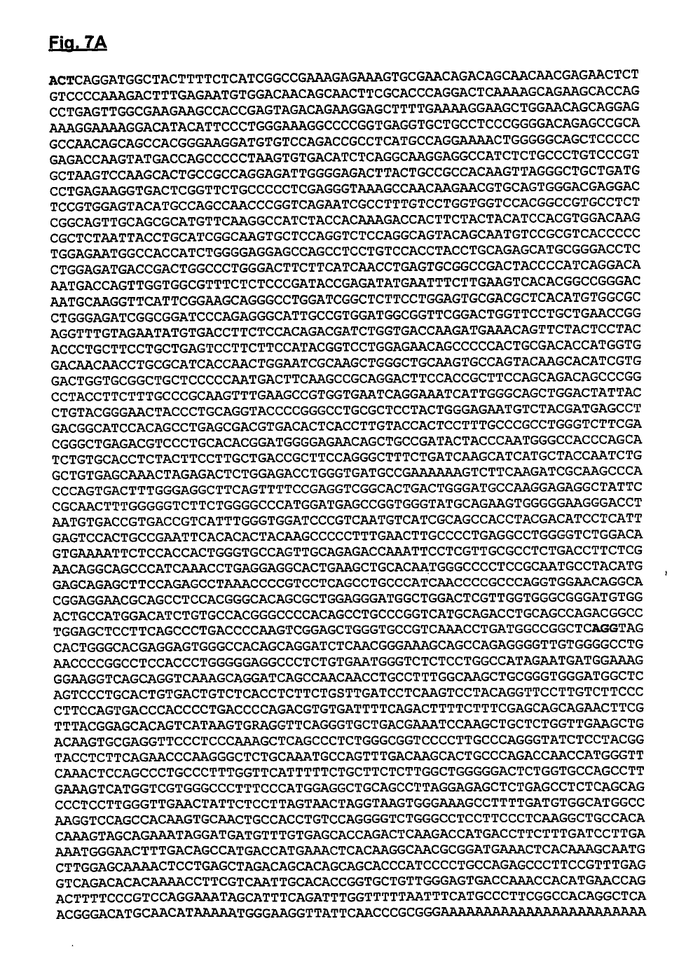

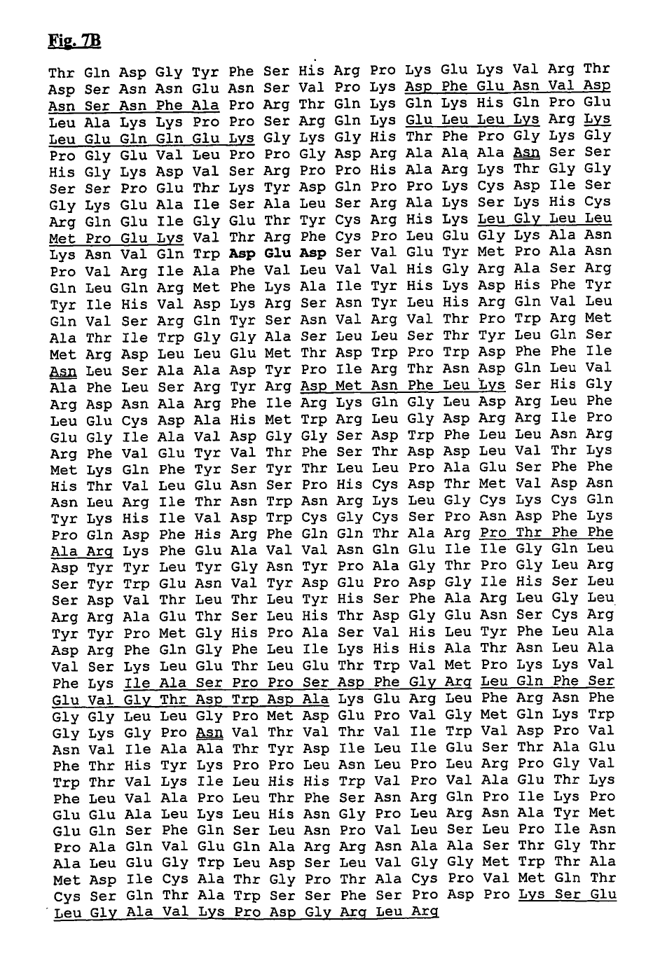

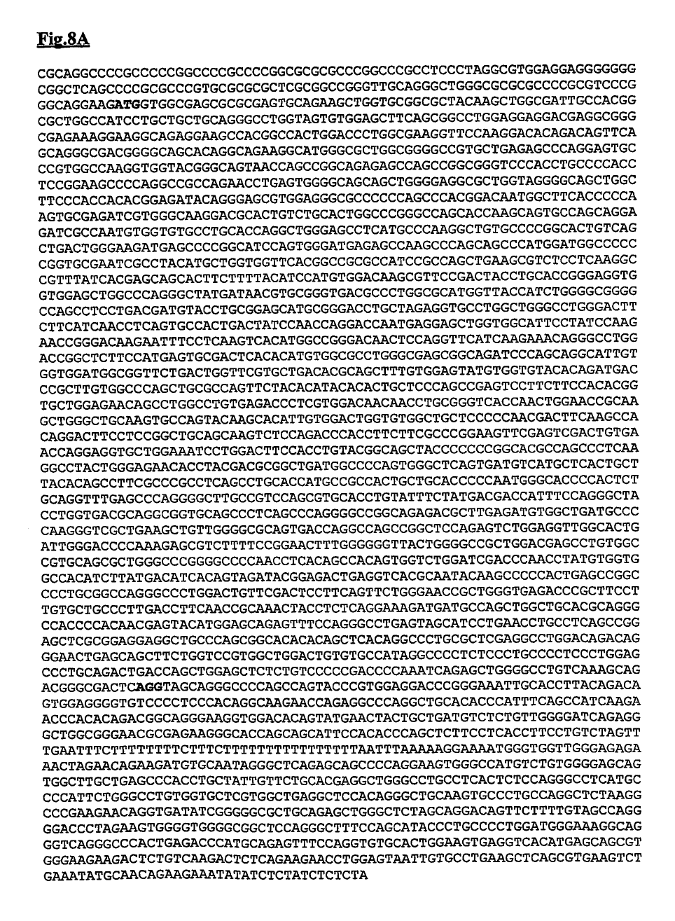

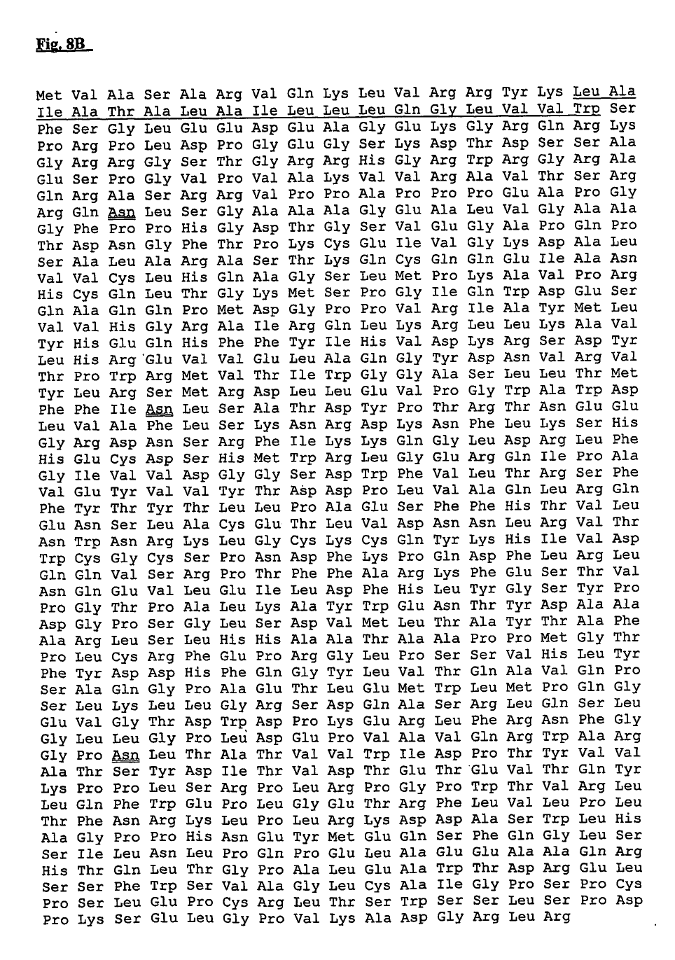

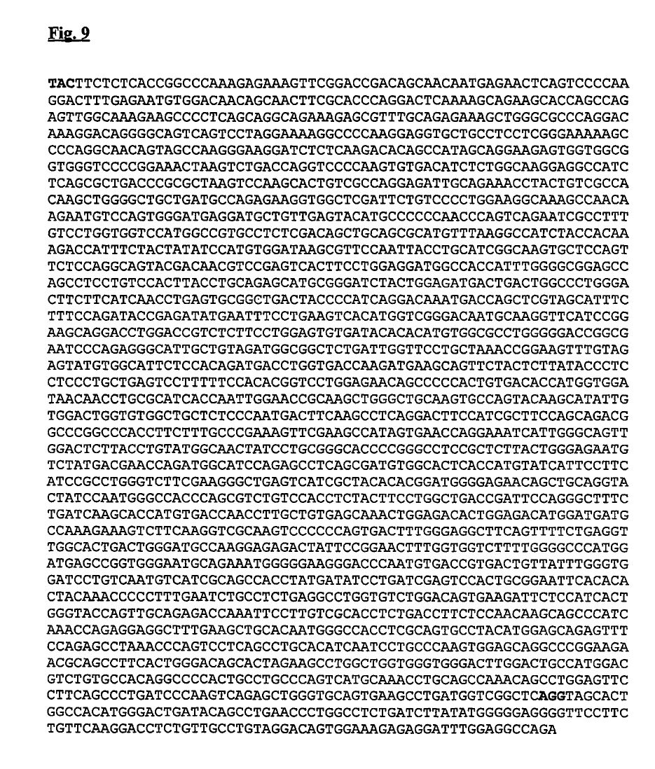

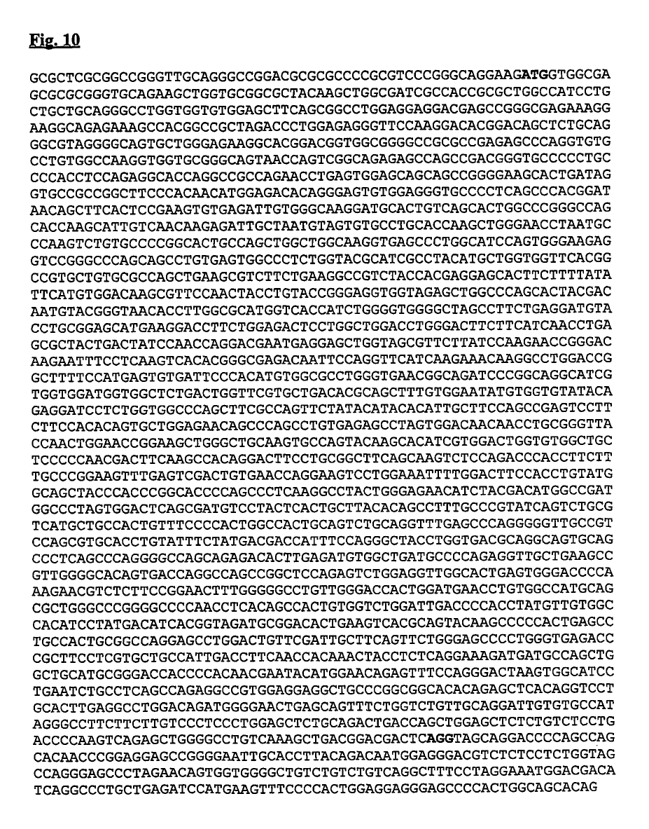

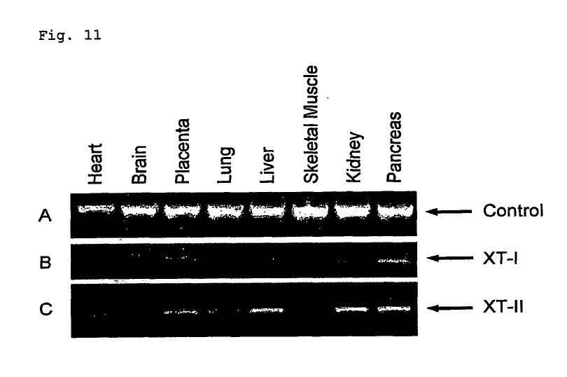

Panel A, Heparin affinity chromatography on POROS 20 HE: The dissolved ammonium sulfate precipitate from JAR choriocarcinoma supernatant was applied to a POROS 20 HE column. After equilibration with buffer A, the column was eluted stepwise with NaCl (------). Protein elution was monitored at wavelength A₂₈₀ (------), and fractions of 38 ml were assayed for XT activity (▪). The horizontal bracket indicates fractions collected for further purification.

Panel B Ion exchange chromatography on!POROS 20HQ: The desalted and concentrated product from heparin affinity chromatography was loaded onto a POROS 20 HQ column. The column was washed with buffer A. Adsorbed proteins were eluted stepwise with NaCl (------). Protein elution was monitored at A₂₈₀ (------), and fractions of 50 ml were assayed for XT activity (▪). The horizontal brackets indicate the fractions collected for further purification. Panel C, Protamine chloride affinity chromatography: The desalted and concentrated product from ion exchange chromatography was applied to a protamine chloride POROS column. After a washing step with buffer A, the column was eluted stepwise with NaCl (------). Elution was monitored at A₂₈₀ (------), and fractions of 6 ml were assayed for XT activity (▪). The horizontal bracket indicates the fractions collected.

FIG. 2 SDS-PAGE of XT fractions at various purification steps

Panel A: XT fractions at various purification steps were subjected to 4-12% gradient polyacrylamide gel for SDS-PAGE. Lane I, JAR cell culture supernatant (crude material); lane II, ultrafiltration retentate; lane III, dissolved ammonium sulfate precipitate; lane IV, protein eluted with NaCl from the POROS 20 HE column; lane V, protein eluted with NaCl from the POROS 20 HQ column; lane VI, protein eluted with NaCl from the protamine chloride POROS column. Lane M, molecular size standard: myosin (200.000), phosphorylase b (97.000), bovine serum albumin (66.000), glutamic dehydrogenase (55.000), carbonic anhydrase (31.000), trypsin inhibitor (22.000). The gel was silver-stained.

Panel B: Collected XT fractions from protamine affinity chromatography were subjected to SDS-PAGE on a 4-12% gradient polyacrylamide gel. The arrows indicate the stained bands corresponding to the unknown protein, which was shown to be XT (120.000) (1), hexose-6-phosphate dehydrogenase (89.000) (2), ezrin (68.600) (3), quiescin Q6 (64.000) (4), plasminogen activator (47.000) (5), aldolase A (39.000) (6) and low molecular artefacts (7). All protein bands were excised, enzymatically digested, and the peptide mixture was characterized by MALDI-TOF mass spectrometry. The gel was stained with Coomassie Brilliant Blue.

FIG. 3: Immunoaffinity chromatography of xylosyltransferase

Panel A 100 μl of the desalted XT-containing fractions eluted from heparin affinity matrix was applied to a column of immobilized polyclonal antibodies. After washing with buffer D, the column was eluted as indicated by the arrow with buffer D/0.15 M NaCl (1) followed by 12 mM HCl (2). Fractions of 1 ml were collected into tubes containing 1 ml of 0.1 M Tris/HCl, pH 8.0. Protein elution was monitored at 280 nm (------) and the XT activities (▪) of each fraction were assayed.

Panel B. Negative control of the immunoaffinity chromatography with immobilized preimmune serum. For conditions see Panel A.

Panel C. Immunoaffinity chromatography with immobilized polyclonal antibodies. The column was eluted with buffer D containing 100 μl (1 mg/ml) of the peptide antigen indicated by the arrow. Fractions of 1 ml were collected into tubes containing 1 ml of 0.1 M Tris/HCl, pH 8.0. Protein elution was monitored at 280 nm (------) and the XT activities (▪) of each fraction were assayed.

FIG. 4: Aprotinin affinity chromatography of partially purified XT and immunoblot analysis of the separated fractions

Panel A 200 μl desalted XT fraction from the heparin purification step was applied to an aprotinin column previously equilibrated with buffer A. After washing with buffer A, the adsorbed proteins were eluted stepwise with NaCl (------). Protein elution was monitored at A₂₈₀ (------) and fractions of 2 ml were assayed for XT activity (▪).

Panel B. Aliquots of fraction 3, fraction 7, fraction 12 and fraction 16 were analyzed by western blot. The MW 120.000 protein was detected in the XT-containing fraction 7 and 12. Immunological detections were performed using a polyclonal rabbit antiserum raised against the synthetic peptide CSRQKELLKRKLEQQEK deduced from the peptides 2 and 10 of the enzymatically cleaved unknown protein. Prestained molecular size standard were myosin (190.000), BSA (64.000), glutamic dehydrogenase (51.000).

FIG. 5: Gelfiltration chromatography and N-glycosidase F digestion

Panel A. 100 μl of the XT containing fraction from heparin affinity HPLC was applied to a TSK G3000 SW column (30 cm×7.5 mm, 10 μl particle size) previously equilibrated with buffer A/0.15 M NaCl. Elution was performed with the same buffer. Fractions of 200 μl were collected and assayed for XT activity (▪). Protein elution was monitored at A₂₈₀ (------). The arrows indicate the elution positions of thyroglobulin (669.000) (1), ferritin (440.000) (2), aldolase (158.000) (3), albumin (67.000) (4), ovalbumin (43.000) (5), chymotrypsinogen A (25.000) (6), and ribonuclease A (13.700) (7).

Panel B. An aliquot of 1 μg of the MW 120.000 protein was analyzed by SDS-PAGE (I). Aliquots (1 μg) of the MW 120.000 protein were digested with 3,1×10⁻³ units of N-glycosidase F at 37° C. for 1 h (II) and 12 h (III). The samples were then subjected to SDS-PAGE, and protein bands were detected by silver staining. The molecular size standard were myosin (200.000), b-galactosidase (116.000), phosphorylase b (97.000).

FIG. 6: Cloning strategy for human XT-I and XT-II isoform.

(A) The peptides 4, 6, 7, and 9 are sequences that were most favorable for the design of degenerate PCR primers. The amino acid sequence of the peptides was obtained after proteolytic digestion of the purified human XT. The strategy for cloning of XT-I cDNA (B) and XT-II isoform (C) is illustrated. The open reading frame of XT-I and XT-II is shown as a filled box, and the location of the peptides 4, 6, 7 and 9 is illustrated by open boxes. The location and orientation of the degenerate primers employed for cloning of XT-I and XT-II are marked by arrows. XT-I and XT-II cDNA inserts contained within the indicated plasmids were obtained using RT-PCR with degenerate primers (PCG111-4, pCG114-29, pCG110-7), 5′ RACE (PCG185-21, pCG212-19, pCG319-23, 3′ RACE (pCG204-38, pCG211-4) and RT-PCR with gene-specific primers (pCG176-1). H=A+C+T, Y=C+T, R=A+G, N=A+G+C+T, I=deoxyinosine.

FIG. 7A. B: Nucleotide (A) and deduced amino acid (B) sequence of the hXT-I.

The position of the peptide sequences obtained by digestion of the purified human XT are underlined (B). Potential N-glycosylation sites are double underlined. The D×D motif is intensified depicted. The figures are identical with SEQ ID 1 and 2.

FIG. 8A, B: Nucleotide (A) and amino acid (B) sequence of the hXT-II isoform.

Potential N-glycosylation sites are double underlined (B). The putative transmembrane domain is underlined. The figures are identical with SEQ ID 3 and 4.

FIG. 9: Nucleotide sequence of rXT-I (SEQ ID 5). The corresponding protein sequence is depicted in SEQ ID 6.

FIG. 10: Nucleotide sequence of rXT-II (SEQ ID 7). The corresponding protein sequence is depicted in SEQ ID 8.

FIG. 11: Differential expression of the XT-I, XT-II gene in human tissues. Semiquantitative RT-PCR with normalized first-strand cDNA was used to examine the abundance of XTI-I and XT-II transcripts. A 983 bp fragment of the housekeeping gene glyceraldehyde-3-phosphate dehydrogenase cDNA was amplified as control (A). The 490 bp XT-I cDNA fragment (B) and the 717 bp fragment amplified from XT-I isoform cDNA (C) were detected in each tissue indicating that both enzymes are ubiquitously expressed. The arrows indicate the expected position for each PCR product.

The following examples describe the invention in more detail without to limit scope of the technical teaching.

### EXAMPLE 1

Materials for Isolation and Purifications. Human JAR choriocarcinoma cells were purchased from ATCC (Rockville, Md.). Dried UltraDOMA-PF medium was obtained from BioWhittaker (Vervier, Belgium) and aqua ad injecta from Braun (Melsungen, Germany). Heat-inactivated fetal calf serum, Dulbecco's phosphate-buffered saline, antibiotic/antimycotic solution, trypsin-EDTA solution, trypan blue, protamine chloride and the Bicinchoninic Acid Protein Assay Kit were purchased from Sigma (Deisenhofen, Germany). Cell culture flasks, serological pipettes, and sterile tubes were purchased from Becton Dickinson (Heidelberg, Germany). The hybrid hollow-fiber bioreactor TECNOMOUSE® was supplied by Integra Biosciences (Fernwald, Germany), the ACA analyzer by Dade Diagnostica (Munchen, Germany) and the Super G analyzer by RLT (Mohnesee, Germany). UDP-[¹⁴C]xylose (9.88 kBq/nmol) came from DuPont (Bad Homburg, Germany), 25 mm diameter nitrocellulose discs from Sartorius (Göttingen, Germany), scintillation mixture and the liquid scintillation counter LS500TD was obtained from Beckman Coulter (Fullerton, Calif.). Ultrafiltration cells, YM1 membranes and PVDF membranes (Immobilon P) were purchased from Millipore (Eschbom, Germany). The chromatography media POROS 20 HQ, POROS 20 HE2, POROS 20 AL, POROS 20 EP and the HPLC workstation Biocad Sprint were supplied by Perseptive Biosystems (Framingham, Mass.). The gel filtration column TSK G3000 SW (30 cm×7.5 mm, 10 μm particle size) was obtained from TosoHaas (Montgomeryville, Pa.). The MALDI mass spectrometer Reflex II was from Bruker Daltonik GmbH (Bremen, Germany) and protein sequencer Procise 494 cLC was purchased from PE Biosystems (Framingham, Mass.). Precast polyacrylamide gels, buffers, and NuPAGE electrophoresis system XCell II Mini-Cell and Blot Module were from Novex (San Diego, Calif.). The synthetic peptide CSRQKELLKRKLEQQEK and the rabbit antiserum were purchased from BioScience (Göttingen, Germany). Peroxidase-conjugated affinpure F(ab′)2 fragment goat anti-rabbit IgG (H+L) was purchased from Dianova (Hamburg, Germany). N-glycosidase F was obtained from Roche (Nannheim, Germany).

### EXAMPLE 2

Cell culture. JAR choriocarcinoma cells releasing XT in the cell culture supernatant were cultured in Ultradoma-PF medium containing 10% heat-inactivated fetal calf serum, 100 U/ml penicillin, 100 μg/ml streptomycin and 250 ng/ml amphotericin B in a humidified atmosphere of 5% CO2 and 95% air at 37° C. After incubation for 24 h in the serum-containing medium, the cell cultivation was adapted to serum-free conditions as described previously (6). Scaling up of XT production was carried out in three hybrid hollow-fiber bioreactors TECNOMOUSE®. During the exponential growth the cells from three 175 cm 2 T-flasks (>3×10⁷cells) were detached with 0.5% trypsin and 0.2% EDTA in Dulbecco's phosphate-buffered saline by incubation at 37° C. for 10 min. After centrifugation (5, min, 1000× g) of the cell suspension the cell pellet was resuspended in 10 ml 37° C. warm serum-free and protein-free Ultradoma-PF medium and washed three times with 20 ml of the same medium. The cell suspension was drawn into a 10 ml syringe and then inoculated into the extracaprnary space (EC space) of the reactor. The bioreactor was connected with a 2 liter medium bottle and set to 150 ml/h in the recirculation mode, and the oxygenation pump was set as described in the operating manual. Five days after inoculation a 10 ml syringe was connected to the left hand EC port and 10 ml of cell culture supernatant was harvested from the EC space. The harvesting was continued every two days over a period of 3 months. Glucose and lactate concentration of the cell culture supernatant were controlled using the Super G analyzer and the ACA analyzer, respectively. The 2 liter medium bottle was replaced every three days. The viability of the cells was determined by trypan blue exclusion.

### EXAMPLE 3

Synthesis of the protamine affinity matrix. Protamine chloride was immobilized as ligand on POROS 20 AL. 30 mg ligand was dissolved in 10 ml of 10 mM phosphate/0.15 M NaCl, pH 7.4. After the protein had been dissolved, 5 ml of 100 mM phosphate/1.50 M NaCl, pH 7.4 was added. NaCNBH₃ was dissolved in the ligand/buffer solution to a final concentration of 5 mg/ml and 1.0 g POROS 20 AL was suspended in the same solution. The suspension was mixed gently on a shaker for 1 minute at RT. An additional 2 ml of 100 nM phosphate/1.50 M NaCl, pH 7.4 was added to the suspension and the mixture was shaken continuously. This step was repeated every five minutes until the mixture volume was 25 ml. After additional shaking for 2 h, the medium was filtered on a sintered glass funnel. The matrix was suspended in 20 ml of 0.2 M Tris/HCl/5 g/l NaCNBH₃, pH 7.2 and mixed gently on a shaker for 30 min at RT. After the media had been washed in a sintered glass funnel using 100 ml of 10 mM phosphate, pH 7.4, 100 ml of 1.0 MNaCl and another 100 ml of 10 mM phosphate, pH 7.4, the matrix was packed in a PEEK column (4.6×50 mm).

### EXAMPLE 4

Synthesis of the aprotinin affinity matrix. Aprotinin affinity matrix was synthesized according to the synthesis of the protamine affinity matrix, but using aprotinin as ligand. After immobilization of the ligand the matrix was packed in a PEEK column (4.6×50 mm).

### EXAMPLE 5

Purification of xylosyltransferase from JAR cell culture supernatant. Fractionated ammonium sulfate precipitation and chromatography steps were performed at RT, ultrafiltration and diafiltration were carried out at 4° C. 18.5 liters JAR cell culture supernatant collected from three hybrid hollow-fiber bioreactors, TECNOMOUSE®, each containing 5 culture cassettes, was concentrated to 800 ml with ultrafiltration cells using YM1 cellulose membranes. The retentate was centriged at 4,000× g for 1 h. The supernatant was decanted, and the pellet was discarded.

Step 1: Fractionated ammonium sulfate precipitation—Solid ammonium sulfate was added to the supernatant to 28% saturation. After 1 h at RT the suspension was centrifuged at 4,000× g for 2 h, the supernatant was decanted, and the precipitate was removed. Additional ammonium sulfate was added to the solution to the point of 40% saturation, and the suspension was allowed to stand for 1 h. To recover the precipitate the supernatant was decanted after the suspension was centrifuged at 4,000× g for 2 h. Before chromatography on immobilized heparin the precipitate was dissolved in 460 ml buffer A (20 mM sodium acetate, pH 6.0).

Step 2: Heparin affinity chromatography on POROS 20 HE2—The step 1 product was passed through a 0.2 μm filter. 4.0 ml of the filtrate was applied to a POROS 20 HE2 column (16×100 mm) equilibrated with buffer A at a flow rate of 40 ml/min. After washing the column with 100 ml of buffer A the XT activity was eluted with the same buffer containing NaCl. The NaCl concentration was increased stepwise: 20 ml buffer A/0.09 M NaCl; 20 ml buffer A/0.15 M NaCl; 30 ml buffer A/0.24 M NaCl; 24 ml buffer A/0.30 M NaCl; 24 ml buffer A/0.60 M NaCl; 24 ml buffer A/1.00 M NaCl; and 24 ml buffer A/1.89 M NaCl. Fractions of 38 ml each were collected and the XT activity was measured. The procedure was repeated 115 times by cyclic chromatography and the fractions containing XT activity (115×38 ml) were collected.

Step 3: Ion exchange chromatography on POROS 20 HQ—Collected fractions from step 2 were desalted using diafiltration with YM1 cellulose membranes and ultrafiltration cells. After concentration of the desalted protein solution to 0.05 liter using analogous techniques the XT-enriched solution was subjected to ion exchange chromatography. 4.0 ml of the XT solution was applied onto the POROS 20 HQ column (16×100 mm) previously equilibrated with buffer A at a flow rate of 40 ml/min. The column was washed with 80 ml buffer A, and the adsorbed protein was eluted stepwise using the same buffer containing 0.07 M NaCl (88 ml), 0.18 M NaCl (120 ml), and 0.36 M NaCl (120 ml) followed by a linear gradient of 0.36-1.00 M NaCl (200 ml) and another step of buffer A/2.0 M NaCl (120 ml). 50 ml fractions were collected and assayed for activity and evaluated by SDS-PAGE. Chromatography was repeated 13 times, and the fractions exhibiting XT activity (13×50 ml) were collected for affinity chromatography.

Step 4: Affinity chromatography on protanine chloride—XT-containing solution from step 3 was desalted as described above and concentrated to 5 ml by ultrafiltration with YM1 cellulose membranes. The ultrafiltration product was passed through a 0.2 μm filter. 100 μl of the filtrate was loaded onto a protamine chloride—POROS column (4.6×50 mm) equilibrated with buffer A. The flow rate was 10 ml/min. The column was washed with 10.0 ml of buffer A, and the adsorbed fraction was eluted with the same buffer containing NaCl by a stepwise increase of the NaCl concentration: 6.6 ml buffer A/0.04 M NaCl; 6.6 ml buffer A/0,06 M NaCl; 6.6 ml buffer A/0.23 M NaCl followed by a linear gradient of 0.23-1.20 M NaCl (4.2 ml) in buffer A. Fractions of 6.0 ml were collected, assayed for XT activity and evaluated by SDS-PAGE. Cyclic chromatography was repeated 50 times. The purified enzyme was collected, concentrated to 1.0 ml using ultrafiltration techniques and stored at −75° C.

Step 5: SDS-Polyacrylamide gel electrophoresis (SDS-PAGE)—The protein composition of various fractions was estimated by SDS-PAGE. Briefly, 12.1 μl of sample was added to 4.7 μl of sample buffer (1.00 M Tris/HCl/1.17 M sucrose, 0.28 M SDS, 2.08 mM EDTA, 0.88 mM Serva Blue G250, 0.70 mM phenol red, 0.10 M DTT, pH 8.5) and heated for 10 minutes at 99° C. After the sample had been loaded, SDS-polyacrylamide gel electrophoresis was carried out on a 4-12% bis-tris polyacrylaride gel with 3-(N-morpholino) propanesulfonic acid (MOPS) running buffer (1.00 M MOPS/Tris/69.3 mM SDS, 20.5 mM EDTA, pH 7.7). Protein bands were detected by Coomassie Brilliant Blue or by silver staining. The Coomassie bands were excised and characterized by MALDI mass spectrometry and amino acid sequence analysis.

### EXAMPLE 6

MALDI mass spectrometry. Coomassie-stained proteins were excised from the gel, repeatedly washed with H₂O and H₂O/acetonitrile and digested overnight with trypsin and endoproteinase Lys-C at 37° C. The peptides generated in the supernatant were analyzed by MALDI mass spectrometry. Sample preparation was achieved following the thin film preparation techniques (13). Briefly, aliquots of 0.3 μl of a nitrocellulose containing saturated solution of a-cyano-4- hydroxycinnamic acid in acetone were deposited onto individual spots on the target. Subsequently, 0.8 μl 10% formic acid and 0.4 μl of the digest sample was loaded on top of the thin film spots and allowed to dry slowly at ambient temperature. To remove salts from the digestion buffer the spots were washed with 10% formic acid and with H₂O. MALDI mass spectra were recorded in the positive ion mode with delayed extraction on a Reflex II time-of-flight instrument equipped with a SCOUT multiprobe inlet and a 337 nm nitrogen laser. Ion acceleration voltage was set to 20.0 kV, the reflector voltage was set to 21.5 kV and the first extraction plate was set to 15.4 kV. Mass spectra were obtained by averaging 50-200 individual laser shots. Calibration of the spectra was performed internally by a two-point linear fit using the autolysis products of trypsin at m/z 842.50 and m/z 2211.10.

### EXAMPLE 7

Amino acid sequence analysis of XT. The MW 120.000 Coomassie-stained protein was excised from the gel, repeatedly washed with H₂O and H₂O/acetonitrile and digested with trypsin and endoproteinase Lys-C overnight. For HPLC separation the excised gel fragment was extracted twice with 0.1% TFA/60% acetonitrile. The extracted enzymatic fragments were separated on a capillary HPLC system equipped with a 140B solvents delivery system (PE Biosystems), Acurate splitter (LC-Packings), UV absorbance detector 759A (PE Biosystems), U-Z capillary flow cell (LC-Packings) and Probot fraction collector (BAI) using reversed-phase column (Hypersil C18 BDS, 3 μm, 0.3×150 mm) and a linear gradient from 12% acetonitrile, 0.1% TFA to 64% acetonitrile, 0.08% TFA in 90 min with a flow rate of 4 μl/min at RT. Peptide elution was monitored at 214 nm and individual fractions from the HPLC separation were analysed by MALDI mass spectrometry. Sequence analysis of separated fragments was performed on a Procise Protein Sequencer 494 cLC using standard programs supplied by PE Biosystems.

### EXAMPLE 8

Determination of XT activity. Determination of XT activity is based on the incorporation of [14C]-D-xylose into the recombinant bikunin according to a previously described method (Brinkmann et al., 1997, J. Biol. Chem., 272, 11171-11175). For analysis of the substrate specificity of the recombinant xylosyltransferases synthetic peptides containing the XT recognition sequence were used as acceptor. The reaction mixture for the assay contained, in a total volume of 100 μl: 50 μl of XT solution, 25 mM 4-morpholineethanesulfonic acid (pH 6.5), 25 mM KCl, 5 mM KF, 5 MM MgCl₂, 5 mM MnCl₂, 1.0 μM UDP-[¹⁴C]-D-xylose, and 1.5 μM of the synthetic peptides. After incubation for 75 min at 37° C., the reaction mixtures were placed on discs (25 mm diameter) of Immobilon-AV membrane, which immobilizes even small peptides by covalent links (Pfund & Bourdage, 1990, Mol. Immunol. 27, 495-502), and allowed to dry. It was then washed for 10 min with 10% trichloroacetic acid and three times with 5% trichloroacetic acid solution. Incorporated radioactivity was determined by liquid scintillation counting.

### EXAMPLE 9

Antiserum Preparation The synthetic peptide CSRQKELLKRKLEQQEK deduced from the sequenced peptides 2 and 10 of the enzymatically cleaved XT was synthesized, purified by HPLC and used for immunization of rabbits (BioScience, Germany). Polyclonal antiserum was obtained by injection of the above antigen followed Example 10:

Preparation of solid-phase antigen. The antigen CSRQKELLKRKLEQQEK was immobilized on POROS 20 EP. After 1.6 mg antigen was dissolved in 1.2 ml of 10 mM phosphate/0.15 M NaCl, pH 7.4 0.60 ml of 100 mM phosphate/1.50 M NaCl, pH 7.4 was added. 400 mg POROS 20 EP was suspended in the solution and the suspension was mixed gently on a shaker at RT. At 10-min intervals, five times in total, an additional 0.24 ml of 100 mM phosphate/1.50 M NaCl, pH 7.4 was added to the suspension. After additional shaking for 5 days at RT the suspension was filtered on a sintered glass funnel. The matrix was suspended in 4 ml 0.2 M phosphate/0.1 M 2-mercaptoethanol, pH 7.4 and mixed on a shaker for 2 h at RT. The matrix was washed in a sintered glass funnel using 20 ml of 10 mM phosphate/0.15 M NaCl, pH 7.4 and 20 ml of 1.0 M NaCl. After additional washing with 20 ml of 10 mM phosphate, pH 7.4, the matrix was packed in a PEEK column (4.6×50 mm).

### EXAMPLE 11

Antibody purification Antiserum was adjusted to 50 mM Tris/HCl, pH 8.0. The solution was clarified by passage through a 0.2 μm filter. 0.4 ml filtrate was applied at 10 ml/min to the antigen column previously equilibrated with buffer B (50 mM Tris/HCl, pH 8.0). After the column was washed with 4.1 ml buffer B and with 12.5 ml buffer B/0.15 M NaCl, the adsorbed antibody was eluted using 12.4 ml of 50 mM sodium citrate/0.15 M NaCl, pH 3.0 followed by 3.4 ml of 100 mM sodium citrate/1.5 M NaCl, pH 3.0. The eluate was collected as 10 ml fractions in tubes containing 2 ml of 0.5 M Tris/HCl, pH 8.0, to immediately neutralize the citric acid.

### EXAMPLE 12

Preparation of immunoaffinity column. Purified antibody was concentrated to a protein concentration of 0.3 mg/ml using ultrafiltration with YM1 cellulose membranes. The antibody solution was adjusted to 10 mM phosphate/0.15 M NaCl, pH 7.4. After filtration of the solution through a 0.2 μm filter 100 μl filtrate was applied at 0.2 ml/min to a POROS 20 PA column (2.1×30 mm). This step was repeated 17 times. Adsorbed antibody was cross-linked using cross-linking solution (100 mM triethanolamine, pH 8.5). After the column was washed with 5 ml of buffer C (10 mM phosphate/0.15 M NaCl, pH 7.4) 2 ml cross-linking solution was applied at 0.5 ml/min onto the cartridge. The procedure was repeated 6 more times, using a total volume of 14 ml cross-linking solution. To block unreacted functional groups on the cross-linking reagents 2 ml of 100 mM monoethanolamine, pH 9.0 (quenching solution), was loaded onto the cartridge at 0.5 ml/min. The column was washed using 2 ml of buffer C and the crossinking step was repeated using another 2 ml quenching solution. The immunoaffinity column was cycled between buffer C and 12 mM HCl/0.15 M NaCl 3 times using a total volume of 12 ml of solution.

### EXAMPLE 13

Immunoaffinity column purification of XT. XT-containing fractions eluted from the heparin affinity matrix were desalted using diafiltration with YM1 cellulose membranes and passed through a 0.2 μm filter. 100 μl of this XT-sample was applied to the immunoaffinity column equilibrated with buffer D (20 mM Tris/HCl, pH 8.0) at a flow rate of 1 ml/min. The column was washed with 1.4 ml of buffer D and with 8.5 ml of buffer D/0.15 M NaCl. The XT activity was eluted with 4.2 ml of 12 mM HCl followed by 1.2 ml of 12 mM HCl/1.5 M NaCl. Alternatively the elution was performed using 100 μl of antigen at 1 mg/ml in buffer D. Fractions (1 ml) were collected into tubes containing 1 ml of 0.1 M Tris/HCl, pH 8.0. The XT activities of the fractions were determined.

### EXAMPLE 14

Aprotinin affinity chromatography. 200 μl of desalted XT solution from the heparin purification step was applied at 10 ml/min to the aprotinin column previously equilibrated with buffer A. After washing the column with 6.6 ml of buffer A the adsorbed protein was eluted stepwise using the same buffer containing 0.3 M NaCl (10.0 ml), 0.54 M NaCl (10.0 ml), 1.00 M NaCl (10.0 ml) and 1.50 M NaCl (2.4 ml). Example 15: Western Blot Analysis. For western blot analysis, proteins were transferred to polyvinylidene difluoride membrane in a semi-dry instrument (Novex). After transfer nonspecific antibody binding sites were blocked with 2% BSA in 0.1 M Tris/HCl, pH 7.2, for 1 h at RT. The membrane was incubated with antiserum in 50 mM phosphate/0.15 M NaCl, 0.5 ml/l Tween 20, pH 7.4 at 1:1000 dilution for 1 h. Bound antibody was detected using a second anti-rabbit goat immunoglobulin coupled to horse-radish peroxidase at a 1:1000 dilution. The blot was developed using 4chloro-1-naphthol.

### EXAMPLE 16

Gel filtration chromatography. A sample of 100 μl from the heparin purification step was applied at 1.0 ml/min to a TSK G3000 SW column (30 cm×7.5 mm, 10 μm particle size) which had previously been equilibrated with buffer A/0.15 M NaCl. Proteins were eluted with the same buffer. Fractions of 200 μl were collected and tested for XT activity. Column calibration was performed using thyroglobulin (669.000), ferritin (440.000), aldolase (158.000), albumin (67.000), ovalbumin (43.000).

### EXAMPLE 17

N-glycosidase F digestion. Aliquots (1 μg) of XT were digested with 3,1×10⁻³ units of N-glycosidase F at 37° C. for 1 h and 12 h (Table II). The samples were then subjected to SDS-PAGE, and protein bands were detected by silver staining.

### EXAMPLE 18

Measurement of protein concentration. Protein concentration was estimated by absorbance at 280 nm assuming E^{1%}_{1 cm}=10.0 or with the Bicinchoninic Acid Protein Assay using bovine serum albumin as a standard.

### EXAMPLE 19

PCR-based cloning of human xylosyltransferase. Degenerate oligonucleotide primers with deoxyinosine substitution were designed based upon the amino acid sequence of peptides obtained after digestion of the isolated human XT with trypsin or Lys-C. The first strand of cDNA was synthesized by the reverse transcription reaction using poly(A)⁺ RNA isolated from the chondrosarcoma cell line SW1353 as template and oligo(dT) as primer. The reverse transcription reaction was performed at 37° C. for 2 h using 50 pmol of oligo(di) primers, 1 82 g of poly(A)⁺ RNA, a 0.5 mM concentration of each DNTP, 1× RT buffer and 200 units of RNase H deficient Moloney murine leukemia virus reverse transcriptase (Life Technologies, Eggenstein, Germany) in a final volume of 20 μl. For PCR amplification the reaction mixture contained 4 μl of the reverse transcription reaction solution, 50 pmol of each primer, a 0.25 mM concentration of each DNTP, 50 mM KCl, 10 mM Tris-HCl (pH 8.3), 1.5 mM MgCl₂ and 2.5 units of hot start Taq polymerase (Life Technologies) in a final volume of 50 μl. Amplification with degenerate oligonucleotide primers was carried out by 40 cycles at 94° C. for 1 min, 48° C. for 1 min, and 72° C. for 2 min, followed by a final extension step at 72° C. for 15 min. After agarose electrophoresis of the PCR products, DNA fragments were excised, subcloned into the pCR2.1 vector (Invitrogen. Groningen, Netherlands) and sequenced by the dideoxy chain termination method using Taq DNA polymerase (Big-dye terminator cycle sequencing kit, Perkin-Elmer, Norwalk, Conn., USA) with an automated DNA sequencer (Applied Biosystems, Weiterstadt, Germany). Several clones were sequenced to compensate for misreading by Taq polymerase.

### EXAMPLE 20

Rapid amplification of 5′ and 3 ′ cDNA ends (RACE). For amplification of the 5′ and 3′ ends of the XT-I and XT-II isoform cDNA RACE experiments were performed using commercially available systems (Clontech, Heidelberg, Germany; Life Technologies) according to the manufacturers' instructions. For 3′ -RACE 1 μg placenta poly(A)⁺ mRNA (Clontech) was reverse transcribed with a 3′-CDS primer (Clontech). PCR amplification of the 3′ cDNA end of human XT-I was accomplished according to a touch-down PCR protocol (5 cycles: 94° C. for 30 s, 72° C. for 3 min, 5 cycles: 94° C. for 30 s, 70° C. for 45 s, 72° C. for 3 min, 25 cycles: 94° C. for 30 s, 65° C. for 45 s, 72° C. for 3 min) with the gene-specific primer GSP1b 5′-GTGGGTATGCAGAAGTGGGGGAAGGGAC-3′ and the UPM primer mix (Clontech). An aliquot of the first PCR was subjected to semi-nested PCR (30 cycles) using the primer Con2111 5′-CCCTCCGCAATGCCTACA-3′ and the UPM primer mix. The DNA fragments obtained were subcloned into the vector pCR2.1 (Invitrogen) and sequenced. PCR amplification of the 3′ cDNA end of the XT-II isoform was carried out with the primers AB10267 5′-ACTGAGGTCACGCAATACAA-3′ and UPMI using the touch-down PCR protocol. For 5′-RACE 1 μg of placenta poly(A)⁺ mRNA was subjected to a reverse transcription and 5′ tailing reaction with the 5′-CDS primer and the SMART oligo (Clontech) according to the manufacturer's protocol. The 5′ end of the XT-I cDNA was amplified using the primer GSP_{—}776L 5′-GCCGCACTCAGGTGATGAAGAAGT-3′ and UPM with a touch-down PCR protocol. An aliquot was used as template in a second semi-nested PCR reaction with the primers GSP_{—}503L 5′-ACCA-CCAGGACAAAGGCGATTCTGA-3′ and UPM. The 5′-cDNA end of XT-II was obtained by 5′-RACE amplification using the primers ABI0315L 5′-AGTCGAACAGTCCAGG-GCCC-3′ and UPM mix. A new primer for a 5′-RACE reaction was designed based upon the nucleotide sequence of the largest fragment obtained (2 kbp), and the experiment was repeated with the primer AB5846L 5′-CACGATCTCGCACTTGGGGG-3′ as described above. The nucleotide sequences of human XT-I and XT-II cDNA have been submitted to the GenBank/EBI Data Bank with the accession numbers AJ277441 and AJ277442.

### EXAMPLE 21

Isolation of XT-I cDNA from human brain and chondrocyte cDNA libraries. Plasmid-DNA was isolated from a human whole brain cDNA library (Life Technologies) and a human chondrocyte cDNA library (Clontech) and used as template in a PCR-based approach for isolation of the 5′ end of the XT-I cDNA. Briefly, GSP776L and 5′ADLD 5′-CTATTCGATGATGAAGATACCCCACCAAACCC-3′ primers were used in a PCR reaction with 1 μg of plasmid DNA as template. The DNA fragments were subjected to agarose gel electrophoresis and 0.7-3 kb long fragments were excised from the gel and used as a template in a semi-nested PCR reaction with GSP_{—}503L and 5′-ADLD primers. DNA fragments were then subcloned into the vector pCR2.1 and sequenced.

### EXAMPLE 22

Cloning of rat XT-I and XT-II cDNA. A RT-PCR based approach using primers based upon the nucleotide sequence of human XT-I and XT-II was employed for amplification of XT-I and XT-II cDNA from rat. The first strand of cDNA was synthesized by the reverse transcription reaction using poly(A)⁺ RNA isolated from the rat liver cell line BRL3A as template and oligo(dT) as primer. DNA fragments obtained after PCR amplification using moderate stringent conditions were subcloned into the vector pCR2.1 and sequenced. The 5′ and 3′ ends of the XT-I and XT-II cDNA from rat tissue were amplified using the RACE strategy with gene-specific primers as described above. The nucleotide sequences of rat XT-I and XT-II cDNA have been submitted to the GenBank/EBI Data Bank with the accession numbers AJ295748 and AJ295749.

### EXAMPLE 23

Expression levels of the XT-I and the XT-II isoform in human tissues. A Human Multiple Tissue cDNA Panel (Clontech) was used for the analysis of expression levels. Levels of amplification of the housekeeping gene glyceraldehyde-3-phosphate dehydrogenase, whose transcript is always present in the tissues at an almost constant level, were determined in parallel for quality control. For amplification of the XT-I encoding transcript the primers 128U and 601L were used, whereas primers AB10267U and AB12394L 5′-GGAAGAGCTGGGTGTGGAAT-3′ were employed for the XT-II. PCR reactions were carried out by 22-36 cycles at 94° C. for 30 s, 60-65° C. for 45 s and 72° C. for 2 min. Amplification of a transcript was performed using a serial number of cycles to find the conditions for semiquantitative amplification, and aliquots were analyzed by agarose gel electrophoresis.

### EXAMPLE 24

Transfection and transient expression of XT. For construction of a eukaryotic expression vector a DNA fragment including the known coding sequence of XT-I cDNA was amplified by PCR using XT_Exp1L 5′-TITCCCGTTGAGATCCTGCT-3′ and XT_Exp3U 5′-ACAGACAGCAACAACGAGAA-3′ as primers and placenta first-strand cDNA (Clontech) as template. The 2450 bp fragment obtained was cloned into the vector pcDNA4/HisMax-TOPO (Invitrogen) resulting in the fusion of XT-I to the Xpress epitope. The plasmid pCG227-XT-I was then transiently transfected into CHO-K1 cells. The coding region of the XT-II cDNA was amplified by PCR using the primers AB_Exp9U 5′-AAAGGAAGGCAGAGGAAGC-3′ and AB_Exp3L 5′-ACCCCTCCACTGT-CTGTAAG-3′ and placenta first-strand cDNA as template. The obtained 2440 bp DNA fragment was cloned into the expression vector pcDNA4/HisMax-TOPO resulting in the fusion of XT-II to the aminoterminal Xpress epitope. The plasmid termed pCG226-XT-II was transiently transfected into CHO-K1 cells. 2×10⁵ cells precultured for 1 day in a 35 mm diameter cell culture dish were transfected with 2 μg of plasmid DNA and 6 μl of Fugene 6 transfection reagent (Roche, Mannheim, Germany). For determination of transfection efficiency CHO-K1 cells were transfected with 2 μg of the control plasmid pcDNA4/HisMax-TOPO-lacZ. 48 h after transfection the cell culture medium was harvested. Protein G agarose beads (Sigma, Deisenhofen, Germany) and mouse anti-Xpress monoclonal antibody (Invitrogen) were added to the cell culture supernatant and incubated at 4° C. for 1 h. After centrifugation at 10.000 g for 1 min the absorbed proteins were twice washed with PBS and resuspended in a final volume of 50 μl. XT activity was then assayed in the samples.

### EXAMPLE 25

Characterization of the reaction products. For characterization of the reaction product of recombinant XT-I the peptide QEEEGSGGGQK, which is homologous to the amino terminus of biknn (Brinkmann et aL, 1997, l.c.), was used as acceptor in the XT activity assay. After incubation for 75 min at 37° C. the enzyme was heat-inactivated by incubation for 15 min at 65° C. and the reaction mixture was used for α- and β-xylosidase treatment and alkaline β-elimination. For the linkage-specific digestion of the bound [¹⁴C]-D-xylose 4 mU of α-xylosidase (Seikagaku Corporation, Tokyo, Japan) or 4 mU of β-xylosidase (Sigma, Dreieich, Germany) were added to the samples and incubated for 60 min at 37° C. The reaction mixtures were then placed on Immobilon-AV membrane discs and allowed to dry. The discs were washed with trichloroacetic acid as described above and the remaining incorporated radioactivity was determined against appropriate controls. The alkaline cleavage of the O-glycosidic linkage between the [¹⁴C]-D-xylose and the β-hydroxyaniino acid serine was performed as described elsewhere (Montreuil et al., 1994). Briefly, the reaction mixture was adjusted to pH 10 with diluted NaOH and an equal volume of cold, freshly prepared sodiumboro-hydride solution (2 M sodiumborohydride in 0.1 M NaOH) and was added. After incubation at 45° C. for 16 h the cooled solution was neutralized by adding 50% acetic acid and placed on Immobilon-AV membrane discs. After drying the discs were washed with trichloroacetic acid and the remaining radioactivity was measured by liquid scintillation counting.

1. An isoform of UDP-D-xylose:proteoglycan core protein β-D-xlosyltransferase(XT).

2. A protein comprising an amino acid sequence of the isoform of claim 1 or a portion thereof, having the biological activity of XT.

3. A protein of claim 1 or 2 deriving from human or rat sources (hXT, rXT)

4. An isolated protein according to claim 3, wherein said hXT has a molecular weight of 120.000 under SDS polyacrlylamide gel electrophoresis conditions.

5. A recombinant protein of claim 3, wherein said protein is hXT-I comprising at least 827 amino acids.

6. A protein according claim 5, having the amino acid sequence as depicted in FIG. 7B.

7. A protein according to claim 3, wherein said protein is hXT-II comprising 865 amino acids and is an isoform of hXT-I.

8. A protein according to claim 7 exhibiting approximately 55% overall sequence identity to hXT-I

9. A protein according claim 8, having the amino acid sequence as depicted in FIG. 8B.

10. A DNA sequence coding for a protein of any of the claims 1-9.

11. A DNA sequence according to claim 10 comprising the nucleotide sequence coding for hXT-I as depicted in FIG. 7A, or rXT-I as depicted in FIG. 9.

12. A DNA sequence according to claim 10 comprising the nucleotide sequence coding for hXT-II as depicted in FIG. 8A or rXT-II as depicted in FIG. 10.

13. An expression vector comprising a promotor sequence, a DNA sequence of claim 10 and optionally a signal sequence.

14. An expression host cell comprising a vector of claim 13, said host cell being capable of expressing a protein of any of the claims 1-9.

15. An antibody directed against a protein as defined in any of the claims 1-9.

16. A process for isolating and purifying a protein as defined in claims 1-4, characterized by the following steps:
(i) culturing cells having an increased level of XT, and harvesting the supernatant of said cell culture,
(ii) fractionated ammonium sulfate precipitation of the supernatant of step (i),
(iii) heparin affinity chromatography of the precipitate of step (ii),
(iv) ion exchange chromatography of the step (iii) product,
(v) affinity chromatography of the step (iv) product, and
(vi) SDS-Polyacrylamide gel elektrophoresis of step (v).

17. A pharmaceutical composition comprising a protein of any of the claims 1-9 and a pharmacologically acceptable carrier, diluent or excipient.

18. Use of a protein of any of the claims 1-9 for the manufacture of a medicament for the treatment of sclerotic diseases and chronic inflammatory joint diseases.

19. Use of claim 18, wherein said medicament is an inhibitor or antagonist of said protein.

20. Use of a protein of any of the claims 1-9 as diagnostic marker.

21. Use of a DNA molecule of any of the claims 10-12 as gene marker.

22. Use of an antibody as defined in claim 15 in an immunological assay for determination of a protein having the biological activity of hXT as diagnostic tool.
